(19) United States
(12) Patent Application Publication
(10) Pub. No.: US 2018/0238915 A1
(43) Pub. Date: Aug. 23, 2018

(54) Microfluidic Platform For Rapid Biologics Analysis

(71) Applicant: Massachusetts Institute of Technology, Cambridge, MA (US)

(72) Inventors: Wei Ouyang, Cambridge, MA (US); Jongyoon Han, Bedford, MA (US)

(21) Appl. No.: 15/752,745

(22) PCT Filed: Aug. 18, 2016
(86) PCT No.: PCT/US16/47567

§ 371 Date: Feb. 14, 2018

Related U.S. Application Data
(60) Provisional application No. 62/206,819, filed on Aug. 18, 2015.

(51) Int. Cl. G01N 33/94; G01N 33/566; G01N 33/557; G01N 33/68; G01N 33/558

(52) U.S. Cl. CPC G01N 33/94; G01N 33/566; G01N 33/558; G01N 33/68; G01N 33/557

(57) ABSTRACT

The invention relates to generally applicable methods and platforms for the assessment of a wide variety of drugs, including biologic drugs. Aspects of the invention further relate to such methods and platforms capable of measuring not only the equilibria but also the kinetics of drug-receptor interactions. Such methods and platforms can be based on mobility-based assays, wherein species are separated along at least one separation dimension. By modulating the mobility, e.g., electrophoretic mobility, of at least one of a drug and a receptor, the separation of drug, receptor, and drug-receptor complex can be optimized, e.g., by enhancing spatial separation of one or more of the foregoing species from the others.

### RELATED APPLICATION

This application claims the benefit of U.S. Provisional Application No. 62/206,819, filed Aug. 18, 2015. The entire teaching of the above application is incorporated herein by reference in its entirety.

### GOVERNMENT SUPPORT

This invention was made with government support under Contract No. N66001-13-C-4025 awarded by the Space and Naval Warfare Systems Center. The government has certain rights in the invention.

### BACKGROUND

Biologics can be less stable than many small molecule drugs, and they can also experience significant batch-to-batch variation during production. Therefore, it is particularly important to ensure the safety and quality of biologic drugs prior to administration to patients. Parameters for equilibrium and kinetic binding of a drug to a suitable molecule, such as a physiologically relevant receptor molecule, can provide an indicator of drug quality or activity, as these parameters can be sensitive to the degradation of the drug. Existing methods of probing such drug interactions (e.g., drug-receptor interactions) include cell-based assays, and ligand binding assays, many of which suffer from one or more limitations. For example, cell-based assays can be time-consuming, taking up to one week to monitor cellular response. Surface plasmon resonance based drug-receptor assays rely on expensive and complex equipment, which limits their application as a point-of-care diagnostic. Technologies that rely on ligand immobilization, e.g., analyte immobilization on the surface of beads, channels or wells, take multiple steps that increase analysis time, can be difficult to implement, and can be subject to factors that limit the sensitivity and reproducibility of the assays, e.g, uncertainties deriving from density and orientation of ligands. Similarly, many microchip assays use ligand immobilization before measurement, thus increasing the analysis time and difficulty in implementation. Many immobilization-free assays, while providing advantages in some regards, can suffer from limited applicability, e.g., inability to measure binding kinetics.

Biologics, such as recombinant hormones and monoclonal antibodies, play a key role in modern medicine. However, due to the inherent complexity and fragility of proteins, manufacturing of biologics can currently take months and require sophisticated facilities, from living-cell based production to cold-chain delivery of biologics. As a result, biologics often fail to reach patients in urgent need in disaster situations and resource-limited settings, while preparedness of biologics for emergencies often results in waster of materials and labor when the threat is not realized.

What is needed, therefore, are new methods and platforms generally applicable to the assessment of a wide variety of drugs, particularly biologic drugs.

### SUMMARY

The invention relates to generally applicable methods and platforms for the assessment of a wide variety of drugs, including biologic drugs. In some embodiments, methods and platforms are based on mobility-based assays, wherein species are separated along at least one separation dimension. For example, modulating the mobility, e.g., electrophoretic mobility, of at least one of a drug and a receptor can enhance the spatial separation of one or more of the drug, the receptor, and a resultant drug-receptor complex. In some embodiments, such as where the mobility assay is combined with electrokinetic concentration, the mobility of the receptor can be modulated such that the receptor is concentratable only upon binding to the drug, and not in its unbound state. Aspects of electrokinetic trapping are described in the article “Million-fold Preconcentration of Proteins and Peptides by Nanofluidic Filter,” by Ying-Chih Wang, Anna L. Stevens, and Jongyoon Han, published in ANALYTICAL CHEMISTRY, Vol. 77, at pages 4293-4299 (2005), and this article is incorporated by reference herein in its entirety.

Significant efforts and great strides have been made in producing therapeutic proteins (TPs) on demand at the point-of-care (PoC) within 24 hours. See, e.g., Choi, E. J.; Ling, G. S. F., Pda. J. Pharm. Sci. Tech 2014, 68 (4), 312-312; and Adamo, A.; Beingessner, R. L.; Behnam, M.; Chen, J.; Jamison, T. F.; Jensen, K. F.; Monbaliu, J.-C. M.; Myerson, A. S.; Revalor, E. M.; Snead, D. R., Science 2016, 352 (6281), 61-67 relevant portions of which are hereby incorporated by reference.

As a part of an integrated end-to-end manufacturing platform, some embodiments related to analytics techniques that can be implemented in-line at PoC and can be used to enable real-time decision-making about the quality of a biologic is highly desirable.

Mobility modulation can be achieved in a variety of ways, such as, for example, by attaching a charged, highly mobile species, such as an oligonucleotide or peptide, to the drug or the receptor. In some embodiments, drug-receptor interactions are determined based on properties of one or more of the detected, spatially separated species, e.g., from signal intensity(ies) integrated across a band. A variety of signals can be used, including signals from a detectable label (e.g., a fluorescent label) or a native property of the drug or receptor (e.g., native fluorescence).

Methods and platforms according to aspects of the invention can be capable of assessing a wide variety of drug aspects, including parameters relating to the kinetics or equilibria, or both, of drug-receptor interactions. In some embodiments, these methods and platforms permit the characterization of aspects of drug-receptor interactions not otherwise observable, e.g., because the mobilities of drug, receptor, drug-receptor complex, or a combination of the foregoing, are not sufficiently different from one another to permit resolution.

An embodiment of the present invention is a method for assaying an analyte using a mobility based assay comprising: providing an analyte and a receptor capable of binding to the analyte to form a complex, the analyte, the receptor, and the receptor-analyte complex having respective mobilities; modifying the receptor to modulate its mobility; combining the analyte and the modified receptor to form a modified receptor-analyte complex; and assaying the combination of the analyte and modified receptor using a mobility based assay to separate species along at least one separation dimension and to detect at least one of the analyte, the modified receptor, and the modified receptor-analyte complex at a respective location along the separation dimension.

In some embodiments, one or more, or any combination of the following is additionally true:

the assaying includes determining at least one of an equilibrium and a kinetic binding parameter for the analyte;

the mobility of the modified receptor has a directionality opposite to that of a mobility of the modified receptor-analyte complex;

the mobility of the receptor has the same directionality as that of the mobility of the receptor-analyte complex;

the mobility based assay is further used to electrokinetically concentrate the modified receptor-analyte complex;

the assay does not provide for an electrokinetic concentration of the receptor in an uncomplexed state.

the receptor is provided with a detectable label;

the analyte is a biologic;

the analyte is a drug;

In other embodiments, one or more, or any combination of the following is additionally true:

the mobility of the modified receptor is greater than, and in the same direction as, the mobility of the receptor;

the mobility of the modified receptor-analyte complex is greater than the mobility of the drug;

the location of the modified receptor-analyte complex along the separation dimension is resolvable from the location of the analyte;

the assaying includes determining at least one of an equilibrium and a kinetic binding parameter for the analyte;

the assaying further comprises providing a labeled analyte, wherein the assaying includes competition between the labeled analyte and the analyte for receptor binding;

the analyte is a biologic;

the analyte is a drug.

Another embodiment of the present invention is a method of determining the activity of a drug comprising: using charge polarity transition or mobility enhancement in a mobility-based assay to determine at least one of a kinetic and an equilibrium binding parameter for a drug-receptor interaction; obtaining a reference value for the at least one of a kinetic and an equilibrium binding parameter; and comparing the determined parameter with the reference value to determine drug activity.

Yet another embodiment of the present invention is a microfluidic drug-receptor binding assay for analyte activity assessment comprising: a receptor conjugated to a charged modulator to form a modulated receptor, the modulated receptor having a net charge, the net charge having a polarity; and an analyte; wherein, a binding of the analyte to the modulated receptor forms a complex having a net charge, the net charge on the complex having a polarity that is opposite to the polarity of the net charge on the modulated receptor.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A illustrates a device for an immobilization-free assay using electrokinetic concentration, according to example embodiments.

FIG. 1B illustrates a schematic of a device, derived from a photograph, for an immobilization-free assay using electrokinetic concentration, according to example embodiments.

FIG. 1C depicts a region of the device from FIG. 1B.

FIG. 1D illustrates principles of an immobilization-free assay using electrokinetic concentration, according to example embodiments.

FIG. 2A illustrates a protein-aptamer interaction using electrokinetic concentration.

FIG. 2B illustrates a protein-protein interaction (between a drug and a receptor) using electrokinetic concentration.

FIG. 3A illustrates principles of a direct assay by positive molecular charge modulation and electrokinetic concentration (MCM-EC), according to example embodiments.

FIG. 3B further illustrates a direct assay by positive molecular charge modulation and electrokinetic concentration (MCM-EC), according to example embodiments.

FIG. 4A illustrates a direct assay by positive molecular charge modulation and electrokinetic concentration (MCM-EC), performed at a first indicated drug concentration, according to example embodiments.

FIG. 4B illustrates a direct assay by positive molecular charge modulation and electrokinetic concentration (MCM-EC), performed at a second indicated drug concentration, according to example embodiments.

FIG. 4C illustrates a direct assay by positive molecular charge modulation and electrokinetic concentration (MCM-EC), performed at a third indicated drug concentration, according to example embodiments.

FIG. 4D illustrates a direct assay by positive molecular charge modulation and electrokinetic concentration (MCM-EC), performed at a fourth indicated drug concentration, according to example embodiments.

FIG. 4E illustrates results of a direct assay by positive molecular charge modulation and electrokinetic concentration (MCM-EC), performed at a range of drug concentrations, according to example embodiments

FIG. 5 shows a semilog plot with drug concentration along the x-axis and fractional binding along the y-axis, according to example embodiments.

FIG. 6A illustrates binding a charged molecule to a receptor to form a high mobility receptor, and a competition between a drug to be tested and a labeled drug for binding to the high mobility receptor, according to example embodiments.

FIG. 6B illustrates a separation of species according to example embodiments.

FIG. 7A illustrates principles of a competitive binding assay, according to example embodiments.

FIG. 7B illustrates principles of separation of species, without competitor, according to example embodiments.

FIG. 7C illustrates principles of separation of species, with competitor, according to example embodiments.

FIG. 7D illustrates a theoretical curve, useful in competition experiments, according to example embodiments.

FIG. 8A is a reaction scheme, showing on and off rate constants, for binding of a labelled drug to a modified, high mobility receptor, according to example embodiments.

FIG. 8B illustrates principles relating to measurement of the dissociation rate of labeled drug from a drug-modulated receptor complex, according to example embodiments.

FIG. 8C illustrates a change in observed signal intensity with time, according to example embodiments.

FIG. 9A shows the experimental results of a direct assay by positive MCM for labeled hGH binding to receptor, according to example embodiments.

FIG. 9B shows further aspects of the experimental results of a direct assay by positive MCM for labeled hGH binding to receptor, according to example embodiments.

FIG. 9C shows further experimental results of a direct assay by positive MCM for labeled hGH binding to receptor, presented as a binding curve, according to example embodiments.

FIG. 10A shows the results of an indirect assay for binding to hGH receptor, illustrating competition between labeled and unlabeled hGH, according to example embodiments.

FIG. 10B further shows results of an indirect assay for binding to hGH receptor, illustrating competition between labeled and unlabeled hGH, according to example embodiments.

FIG. 10C shows a binding curve, relating to results of an indirect assay for binding to hGH receptor, illustrating competition between labeled and unlabeled hGH, according to example embodiments.

FIG. 11A shows the experimental results of a competitive assay by negative MCM for IFN binding to receptor, according to example embodiments.

FIG. 11B shows the experimental results of a competitive assay by negative MCM for IFN binding to receptor, presented as a semilog plot, according to example embodiments.

FIG. 12A shows the experimental results of a competitive assay by negative MCM for GCSF binding to receptor, according to example embodiments.

FIG. 12B shows the experimental results of a competitive assay by negative MCM for GCSF binding to receptor, presented as a semilog plot, according to example embodiments.

FIG. 13A shows the evolution of the peak of receptor-drug complex for hGH, according to example embodiments.

FIG. 13B shows the evolution of the peak of receptor-drug complex for IFN, according to example embodiments.

FIG. 13C shows the evolution of the peak of receptor-drug complex for Insulin, according to example embodiments.

FIG. 13D shows the evolution of the peak of receptor-drug complex for G-CSF, according to example embodiments.

FIG. 13E is a chart summarizing off-rates for receptor-drug complex according to example embodiments, and with comparison values.

FIG. 13F depicts as a bar chart the dissociative half-life for hGH, IFN, G-CSF and Insulin complexes, determined according to example embodiments.

FIG. 14A shows the results of evaluating hGH after exposure to thermal stress, according to example embodiments.

FIG. 14B shows another representation of data relating to results of evaluating hGH after exposure to thermal stress, according to example embodiments.

FIG. 14C depicts relative activity for HGH as a function of temperature.

FIG. 14D shows the results of evaluating G-CSF after exposure to thermal stress, according to example embodiments.

FIG. 14E shows another representation of data relating to results of evaluating G-CSF after exposure to thermal stress, according to example embodiments.

FIG. 14F depicts relative activity for G-CSF as a function of temperature.

FIG. 15A shows the results of evaluating hGH after exposure to light, according to example embodiments.

FIG. 15B shows another representation of data relating to results of evaluating hGH after exposure to light, according to example embodiments.

FIG. 15C depicts relative activity for HGH as a function of light exposure.

FIG. 15D shows the results of evaluating G-CSF after exposure to light, according to example embodiments.

FIG. 15E shows another representation of data relating to results of evaluating G-CSF after exposure to light, according to example embodiments.

FIG. 15F depicts relative activity for G-CSF as a function of light exposure.

FIG. 16A shows the results of evaluating hGH after exposure to oxidative stress, according to example embodiments.

FIG. 16B shows another representation of data relating to results of evaluating hGH after exposure to oxidative stress, according to example embodiments.

FIG. 16C depicts relative activity for HGH as a function of exposure to oxidative stress.

FIG. 16D shows the results of evaluating G-CSF after exposure to oxidative stress, according to example embodiments.

FIG. 16E shows another representation of data relating to results of evaluating G-CSF after exposure to oxidative stress, according to example embodiments.

FIG. 16F depicts relative activity for G-CSF as a function of exposure to oxidative stress.

FIG. 17A shows the results of evaluating hGH at extended times, according to example embodiments.

FIG. 17B shows another representation of data relating to results of evaluating hGH at extended times, according to example embodiments.

FIG. 17C depicts relative activity for HGH as a function of extended time.

FIG. 17D shows the results of evaluating G-CSF at extended times, according to example embodiments.

FIG. 17E shows another representation of data relating to results of evaluating G-CSF at extended times, according to example embodiments.

FIG. 17F depicts relative activity for G-CSF as a function of extended time.

FIG. 18 is a chart of calculated charges of mobility modulators, according to principles of the present invention.

FIG. 19 is a chart of mobility versus degree of conjugation, according to principles of the present invention.

FIG. 20A depicts degrees of oxidation of the indicated methionine residues for oxidized hGH samples.

FIG. 20B depicts degrees of oxidation of the indicated methionine residues for oxidized G-CSF samples.

FIG. 20C shows results of testing the oxidized hGH by MCM-EC based assays.

FIG. 20D shows results of testing the oxidized G-CSF by MCM-EC based assays.

FIG. 20E shows dose-response curves and relative potency of oxidized hGH in cellular bioassay.

FIG. 20F shows dose-response curves and relative potency of oxidized G-CSF in cellular bioassay.

FIG. 21A depicts sequence information of material referred to herein.

FIG. 21B depicts sequence information of material referred to herein.

FIG. 21C depicts sequence information of material referred to herein.

### DETAILED DESCRIPTION

A description of example embodiments of the invention follows.

### Definitions

As used herein, the term “analyte” means and includes a variety of chemical species, including small molecules, biomolecules, and macromolecules. Some analytes are drugs, e.g., biologics. Some analytes are therapeutic proteins (“TP”), clinically relevant members of which can include recombinant hormones and monoclonal antibodies. hGH, IFNα2b, and G-CSF represent three important categories of TPs—recombinant hormones, interferons and growth factors, respectively

As used herein, the term “receptor” means a substance to which the analyte binds, and includes, without limitation, proteins, peptides, DNA, RNA, and oligonucleotides. Receptors that bind therapeutic proteins (“TP”) are therapeutic protein receptors (“TPR”).

The binding of a receptor to an analyte can be avid, specific, or both. Some receptor-analyte interactions are characterized by equilibrium dissociation constants of less than about 1 μM, 100 nM, 10 nM, 1 nM, 100 pM, 10 pM, or 1 pM. The interaction of a receptor and an analyte can be followed by further events, such as, without limitation, conformation change of the receptor, signal transduction, and downstream signaling.

As used herein, the term “mobility modulator” refers to a substance that can modulate the mobility of a receptor or analyte by interacting with it, such as by binding or covalent attachment, to form a complex or combination. Mobility modulators can include a wide variety of substances, including oligonucleotides, proteins, and charged polymers. Mobility modulators can alter the mobility of the receptor or analyte in a variety of ways, such as by altering the charge, mass, or shape of the target or analyte. A mobility modulator can be selected to achieve a desired mobility of the modulated species, e.g., a modulated receptor. The mobility of a species, e.g., electrophoretic mobility, is related to physical properties of the species, such as charge, mass, volume, according to well-known equations. Mobility modulators that alter the net charge of a species (e.g., receptor) upon binding are referred to as charge modulators.

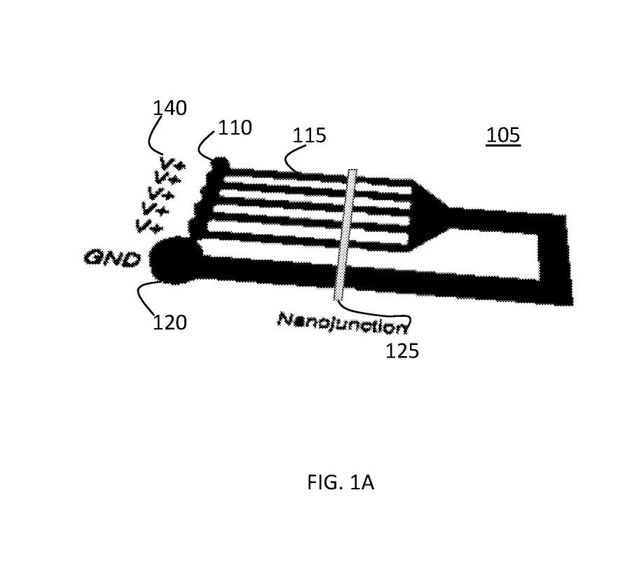

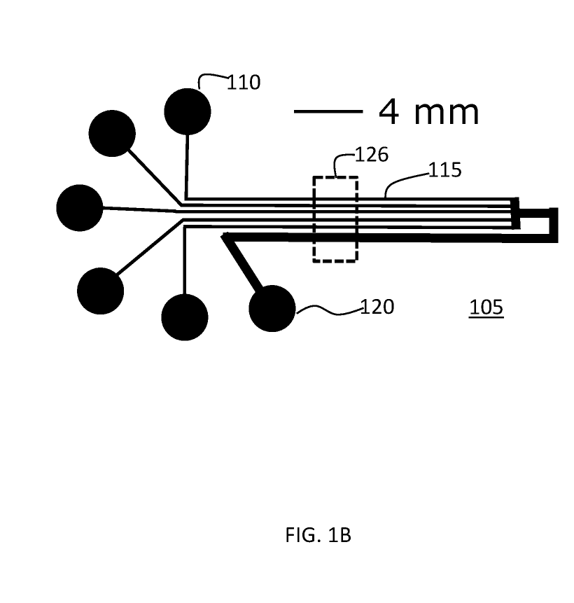

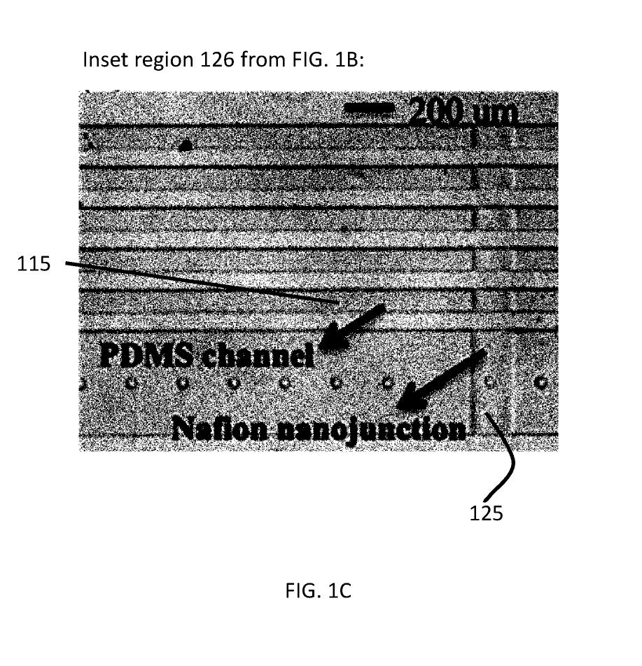

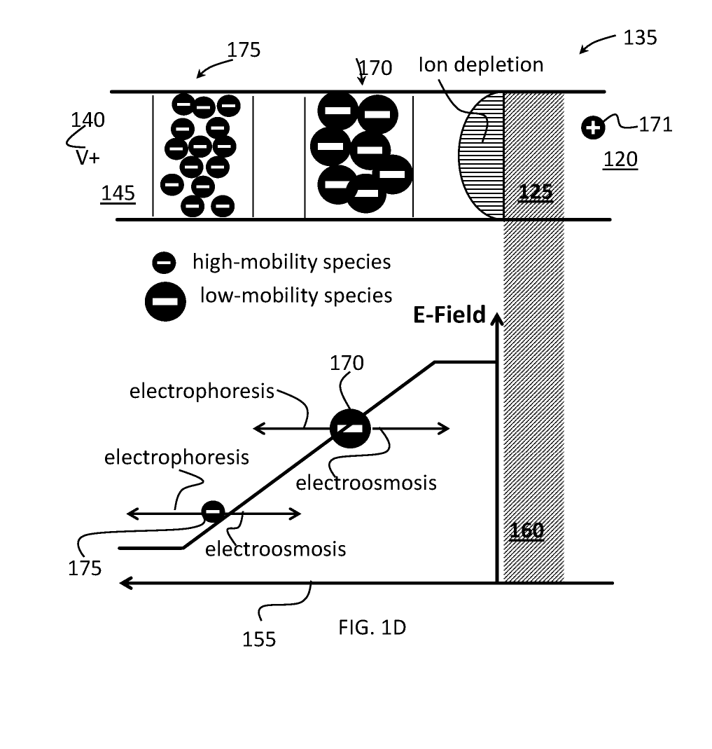

FIG. 1A illustrates a mobility-based assay device 105 usable in connection with embodiments of the present invention. Device 105 has five inlets 110 with corresponding microchannels 115 and one common outlet 120, allowing five samples at be analyzed at the same time. A DC voltage 140 is applied between the inlets 110 and the outlet 120, which generates an electric field. Samples applied at inlets 110, e.g., an equilibrium solution of drug, receptor, and drug-receptor complex, wherein one or more of the species have been mobility-modulated, can be spatially separated based on electrophoretic mobility across a separation dimension, corresponding to microchannels 115. It should be appreciated that the applied voltage can also generate an electroosmotic flow along the microchannels. The distribution of species along the length of microchannels 115 can depend on the velocity of the electroosmotic flow, the velocity of the electrophoretic flow, and the elapsed time.

The device of FIG. 1A further contains a cation-selective nanojunction 125 between the inlets 110 and the outlet 120. FIG. 1A is an example of an electrokinetic concentrator. The electric field is nonuniform across the separation dimension. Nanojunction 125 contributes to ion concentration polarization and to the electric field in the vicinity of the nanojunction. Negatively charged species upstream of the membrane are influenced by the locally enhanced field as depicted in FIG. 1D, infra.

FIG. 1B is a schematic derived from a photograph of an electrokinetic concentrator, showing microchannels 115, inlets 110, and outlet 120. Fabrication of electrokinetic concentrators can be performed by a variety of techniques, such as standard microfabrication and poly(dimethylsiloxane) (PDMS) molding techniques. Inset region 126 is indicated, a magnified view of which is provided by FIG. 1C.

FIG. 1C shows a magnified view of inset region 126, showing nanojunction 125 and microchannels 115. Nanojunction 125 is a cation-selective nafion nanojunction patterned on a glass slide by a microflow technique. It should be appreciated that a wide variety of nanojunction materials can be used, including anion-selective nanojunctions.

FIG. 1D schematically illustrates aspects of device 105 in FIGS. 1A and 1B within one microchannel 135 of FIG. 1A's five microchannels 115. When a DC voltage 140 is applied between the inlets (110, 145) and the outlet 120, an electric field is created across the length of the microchannel. The electric field increases in magnitude near the nanojunction 125 as shown. When sample is applied at the inlet end (left of figure), species will move as dictated by a combination of electrophoretic and electroosmotic influences. For the system shown, electroosmotic flow is from left to right, consistent with a negative surface charge on the walls of the microchannel. The migration velocity for a given species will reflect the contributions of both electroosmotic velocity and electrophoretic velocity. Species will concentrate at a region along the separation dimension 155 (microchannel 135) where the net velocity is zero, i.e., where the electroosmotic and electrophoretic components are equal and opposite.

Species 175 and 170, as shown in FIG. 1D, differ in their mobility. Both species are depicted as having a single negative charge, but the high mobility species is drawn smaller, reflecting a smaller effective volume/cross-sectional area, and therefore a higher mobility. Because electrophoretic migration velocity relates to the product of the local electric field and the mobility, the high mobility species 175 requires less of an electric field than does low mobility species 170 in order to prevent it from being swept downstream by electroosmotic flow. Accordingly, the high mobility species localizes farther from the nanojunction 125/160.

Also depicted in FIG. 1D is positively charged species 171, which is unconcentratable by the depicted device because it flows past the nanojunction, driven by the electric field.

It should be appreciated that the magnitude and directionality of electroosmotic flow can be manipulated in a variety of ways well-known in the art, including by varying the buffer composition or pH, and the application of coatings to, or use of different materials for, microchannel walls, thereby reversing the polarity of the electrical double-layer. Reversing the polarity of the external field will also reverse the direction of electroosmotic flow. It should likewise be appreciated that the magnitude and directionality of electrophoresis for a given species will depend on the electric field strength and orientation amongst other factors, e.g, charge on the species, mass, and effective volume.

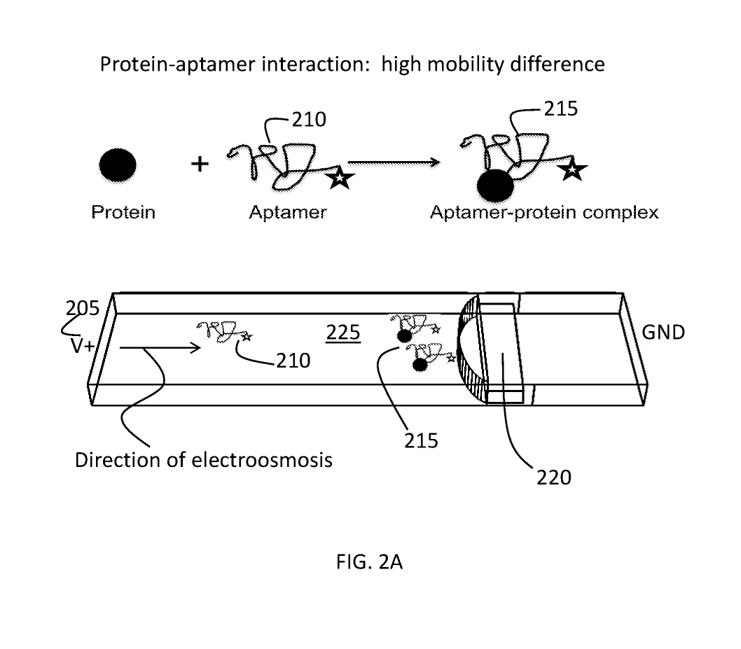

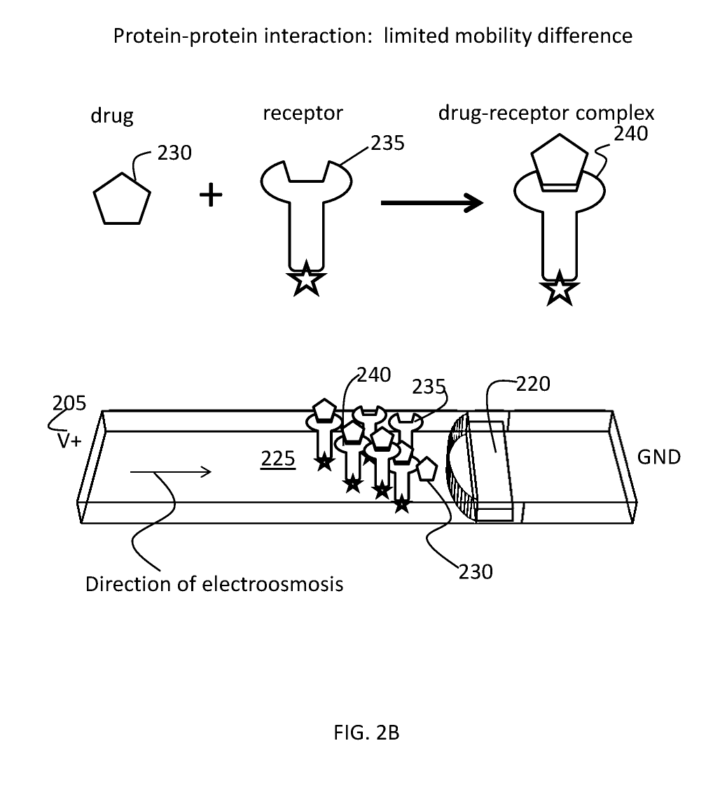

FIGS. 2A and 2B illustrate the significance of mobility differences among interacting species in probing these interactions using a mobility-based assay. FIG. 2A depicts an interaction in an electrokinetic concentrator between species that have a high inherent mobility difference: fluorescently labeled aptamer 210 and aptamer-protein complex 215. When voltage 205 is applied across the cation-selective nanojunction 220, the high inherent mobility difference between the protein-aptamer complex 215 and aptamer 210 spatially separates these species within the microchannel 225 of the electrokinetic concentrator. As a result, the fluorescence tags bound to the aptamer are separated in a bound and unbound state, permitting resolution of binding parameters using the assay.

Aspects of aptamer-protein interactions are described in the article entitled “Continuous Signal Enhancement for Sensitive Aptamer Affinity Probe Electrophoresis Assay Using Electrokinetic Concentration,” by Lih Feng Cheow and Jongyoon Han, published in ANALYTICAL CHEMISTRY, Vol. 83, at pages 7086-7093 (2011), and this article is incorporated by reference herein in its entirety. Aspects of electrophoretic mobility shift assays are discussed in the article entitled “Microfluidic electrophoretic mobility shift assays for quantitative biochemical analysis,” by Yuchen Pan, Kelly Karns, and Amy E. Herr, published in ELECTROPHORESIS, Vol. 35, at pages 2078-2090 (2014), and this article is incorporated by reference herein in its entirety. See also “Detecting kinase activities from single cells using concentration-enhanced mobility shift assay,” Lih Feng Cheow, Aniruddh Sarkar, Sarah Kolitz, Douglas Lauffenburger and Jongyoon Han, Analytical Chemistry, 86(15), 7455-7462 (2014), which is also incorporated by reference herein in its entirety.

FIG. 2B illustrates probing the interaction of species having a low mobility difference within an electrokinetic concentrator. The mobility of drug 230 and receptor 235 are similar, and, as a consequence, are similar to the mobility of resultant complex. Many proteins have similar mobilities. When both drug and receptor are proteins, problems for mobility-based drug assays can arise. Inherent mobility difference between the bound and unbound species can be insufficient to establish spatial separation in a mobility assay.

As shown, unlabeled drug 230 binds to labeled receptor 235 and forms a drug-receptor complex 240. The inherent mobility difference between the unbound species (230 and 235) and drug-receptor complex 240 is low. When voltage 205 is applied across the cation-selective nanojunction 220, the low mobility difference between the bound (240) and unbound (230 and 235) species is insufficient to spatially separate the bound drug-receptor complex 240 from the unbound drug 230 and receptor 235. Therefore, both the bound (240) and unbound (230 and 235) species concentrate near the nanojunction 220, and the unbound and labeled receptor 235 is indistinguishable from the bound and labeled drug-receptor complex 240. In some cases, electrophoretic assays, while offering immobilization free measurement of K_{D} and k_{off} by mobility-based separation of bound and unbound species can be limited by the fact that biomolecules binding sometimes does not result in resolvable mobility differences, even at higher voltages, e.g., greater than 1 kV.

As illustrated in subsequent figures, some embodiments of the invention relate to modulating the mobility of at least one of an analyte (e.g., a drug, a biologic, or small molecule) and a receptor prior to performing a mobility-based assay in order to provide, without limitation, for greater spatial separation of species in the assay, to selectively concentrate one or more species during the assay, or both. In some embodiments, the modulating is performed by adjusting the charge on the receptor by conjugation with charged molecules. In other embodiments, mobility is modulated by changing other mobility contributors, e.g., effective volume, shape, mass.

In some embodiments, mobility modulators are selected so that they have no effect, a minimal effect, or a known effect, on the binding of drug to the receptor. In some embodiments, the binding affinity of drug for receptor differs from its affinity for modulator-receptor complex by less than an order of magnitude, and in some embodiments is equal thereto. In some embodiments, the on-rate constant, off-rate constant, or both for drug binding to receptor differ from those for modulator-receptor complex by less than an order of magnitude, and in some embodiments are equal thereto.

Assays performed as described herein can have advantages over current assays. In some embodiments, the binding and separation is achieved in bulk solution and in an immobilization-free manner. In some embodiments, assays have a short operation time, e.g., thirty minutes of incubation and between one to thirty minutes of testing in the device, depending on the type of assay. In a further embodiment, small sample volumes of a few micro-liters are needed. In some embodiments, the detection of the binding and separation of biomolecules can be performed by an on-line application and allows a biologic to be assessed and released for use in real-time. In some embodiments, devices have a low fabrication cost. For example, a PDMS device can be fabricated at very low cost. Silicon devices also have a low fabrication cost and can be reused for a few years. In addition to biologics, various biomolecules such as DNA, RNA, and peptide, can be analyzed.

In some embodiments, assays can provide rapid assessment of the binding affinity and binding kinetics of biologics, which are important parameters in the quality control of biologics production. In some embodiments, portable platforms for the production of on-demand biologics product can overcome the shortage of biologics in developing countries. In some embodiments, a platform is provided for on-line quality control and release of biologics for real-time use. In some embodiments, the degradation of biologics during transport and storage can be detected by this platform with diagnostics at points-of-care before final administration in patients.

According to some embodiments of the present invention, bioactivity (a key quality attribute) of analytes (e.g., TPs) can be assessed. The bioactivity assessment may include assessment of equilibrium dissociation constant (K_{D}) of analyte from receptor (e.g., of TP from TPR), or the kinetics of the same process, such as equilibrium dissociation rate constant, k_{off}.

In some embodiments, various molecular charge modulation strategies can be used, including mobility-enhancement strategies and charge polarity transition strategies.

In some embodiments, an immobilization-free (homogeneous), yet generally applicable platform, is used for the rapid assessment of TPs' activity based on molecular charge modulation (MCM) and electrokinetic concentration (EC), as more fully described below. EC can, in some embodiments, simultaneously concentrate and separate bound and unbound species in an assay provided that they have a sufficient mobility difference. MCM can, in some embodiments, enable the application of EC to a wide variety of TPs by artificially enhancing the mobility difference despite the intrinsic mobility of TPs, TPRs and their complexes.

The utility of this platform was demonstrated in a variety of ways, including way by analyzing three TPs with distinct equilibrium and kinetic binding behaviors—human growth hormone (hGH), interferon alpha 2b (IFNα2b), and granulocyte colony stimulating factor (G-CSF), which represent three important categories of TPs—recombinant hormones, interferons and growth factors, respectively. In these experiments, the platform was capable of assessing the activity of TPs and providing results conforming well to those obtained from other technologies, but with significantly less time (e.g., <1 h) and simpler experimental setup.

By coupling the nanojunction-induced ion depletion effect with electroosmosis in a microchannel, EC can be used in some embodiments to simultaneously concentrate and separate a wide variety of bound and unbound species having a sufficient mobility difference in an assay. In some embodiments of the present invention, the scope and utility of EC assays is expanded by MCM, which can augment the separation resolution of EC by artificially enhancing the mobility difference with mobility modulators, despite the intrinsic mobility of TP, TPR and TP-TPR complex.

MCM includes, without limitation, the techniques of charge polarity transition strategies (e.g., positive MCM (pMCM)) and mobility enhancement strategies (e.g., negative MCM (nMCM)).

Charge Polarity Transition Strategies (e.g., pMCM)

One example of a molecular charge modulation strategy is a polarity transition strategy. Accordingly, in some embodiments, the net charge on the receptor is modulated such that the sign of the net charge changes upon binding to drug. For example, if drug is negatively charged, the charge on the receptor, such as for example a negative charge at buffer pH, is tuned by adding enough positively-charged polypeptide such that the net charge on the modified-receptor is positive, while the net charge on the modified receptor-analyte complex is negative. As such, the directionality of the electrophoretic velocity for modulated receptor bound to drug is opposite that of unbound modulated receptor. In some embodiments, such as where electrokinetic concentration is used, only one of those species is concentratable.

A charge polarity transition strategy, specifically by positive molecular charge modulation, can be used, for example, to probe the interaction on an EC of TPs and TPRs (proteins), which are often negatively charged at neutral pH. In some embodiments, for example, each TPR molecule can be conjugated with one positively charged fluorescent peptide molecule such that the following is true: Q(peptide)+Q(TPR) >0, Q(peptide)+Q(TPR)+Q(TP)<0, where Q refers to electric charge. In this manner, while the TPR-peptide conjugate (pTPR) is positively charged and unconcentratable by EC (due to the cation-selectivity of the Nafion nanojunction), upon binding, the TP-pTPR complex becomes negatively charged and is concentrated. By monitoring the presence and intensity of the concentration band, the TP-pTPR binding can be quantified, yielding K_{D} measurement.

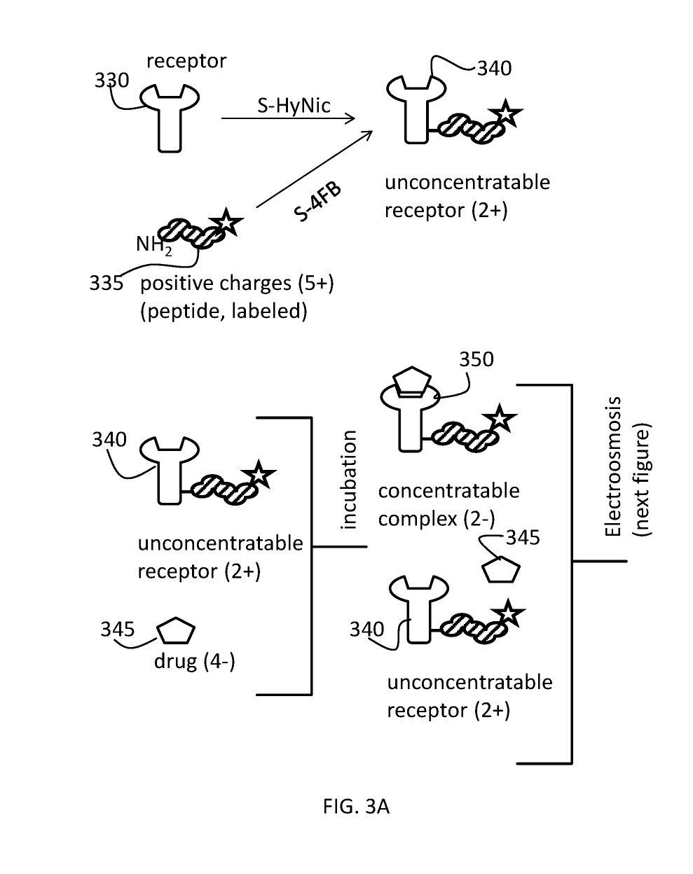

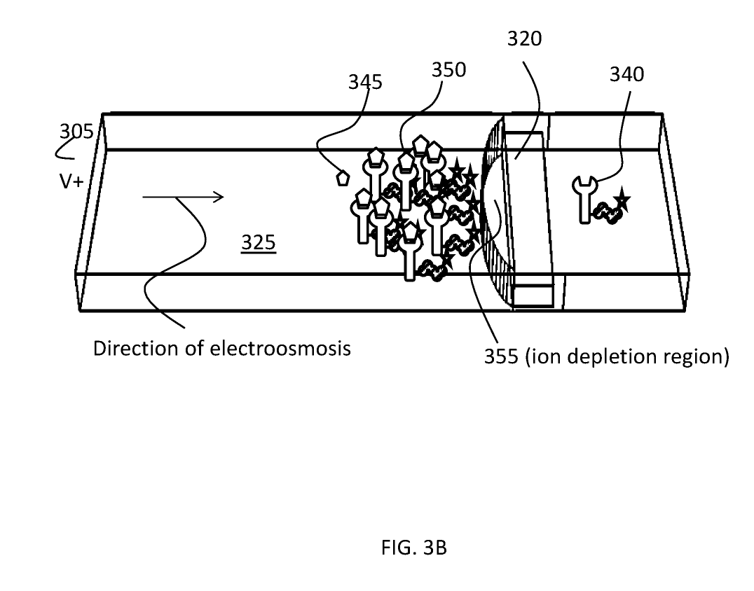

FIG. 3A illustrates a direct assay by charge polarity transition, using positive molecular charge modulation (“MCM”) and electrokinetic concentration (“EKC”). Fluorescently labeled peptides with net positive charge 335 are used as mobility modulators. Peptide 335 is conjugated to receptor 330. Receptor 330 is depicted as having no net charge. The resulting labeled receptor-peptide pair 340 has a net positive charge and is unconcentratable with electrokinetic concentration. When negatively charged drug 345 (e.g., a biologic, a DNA or RNA drug, or small molecule drug) binds to receptor-peptide pair 340, the resultant complex 350 bears a net negative charge 350 and is concentratable with electrokinetic concentration.

Further shown in FIG. 3B is the introduction of a preincubated (e.g., pre-equilibrated) mixture of drug 345 and receptor-peptide pair 340 into a microchannel 325 of the electrokinetic concentrator. The mixture contains complex 350, free receptor-peptide pairs 340, and free drug 345. A voltage 305 is applied across the cation-selective nanojunction 320, and an ion depletion area 355 forms on one side of the cation-selective nanojunction 320. The unconcentratable receptor-peptide pairs having a net positive charge 340 pass the cation-selective nanojunction 320. However, the ion depletion area 355 prevents the receptor-drug complex 350 and unbound drug 345 from passing the cation-selective nanojunction. The labeled receptor-drug complex 350 and the unbound and unlabeled drug 345 are concentrated close to the ion depletion area 355.

It should be appreciated that if the receptor has a net negative charge at the pH of the run buffer, then the net charge on peptide mobility modulator 335 can be chosen so that the net charge on the modulator-receptor complex is positive (unconcentratable in the device as shown, with cation-selective membrane) and the net charge on the modulator-receptor-drug complex is negative (concentratable). Likewise, if the receptor has a sufficient net positive charge at the pH of the run buffer that the drug-receptor complex is also positively charged, then mobility modulator 355 can be selected to impart a net negative charge (e.g., an oglionucleotide, or a polypeptide with negatively charged side chains at pertinent pH). The positive charge on the receptor is then tuned so that it is only slightly positive, such that binding to drug results in a complex of opposite (negative) charge, and that is concentratable.

In should be further appreciated that if the polarity of the applied electric field is reversed, then negative species will be concentratable and positive species unconcentratable, and the method will involve tuning molecular charge in opposite directions to achieve analogous results. In this case, a negative MCM could be used to effect a desired charge polarity transition experiment.

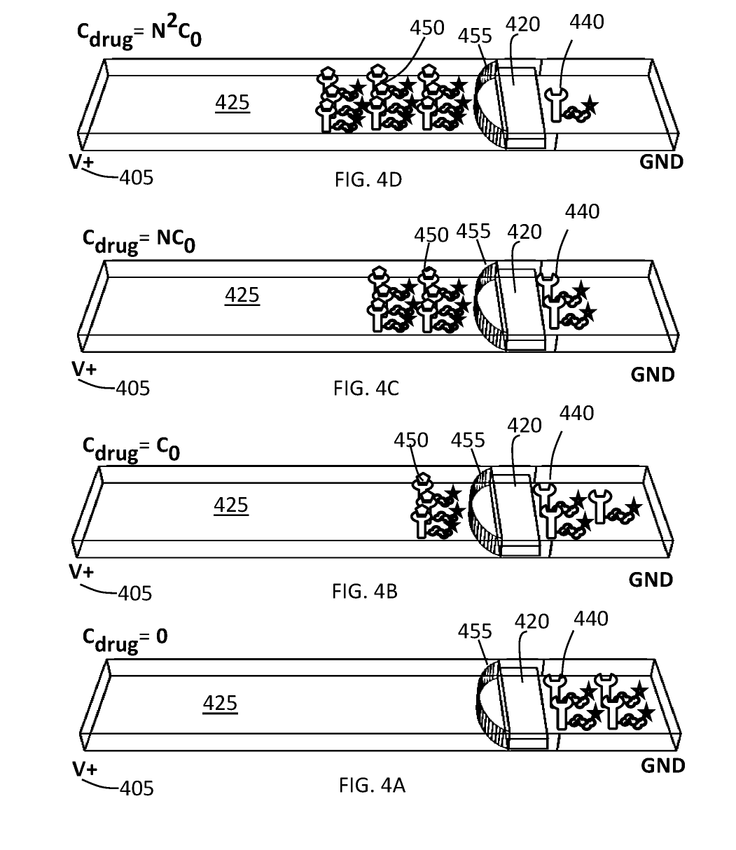

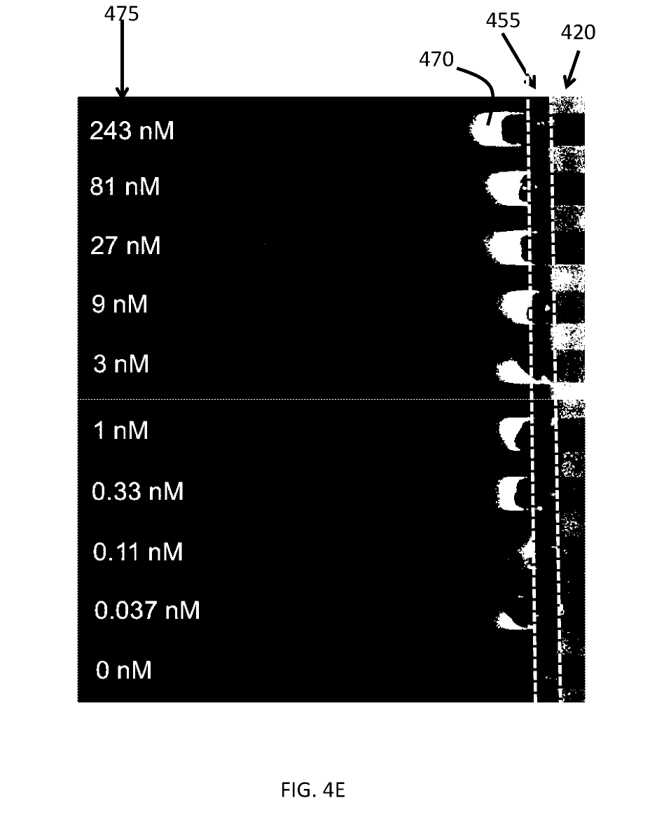

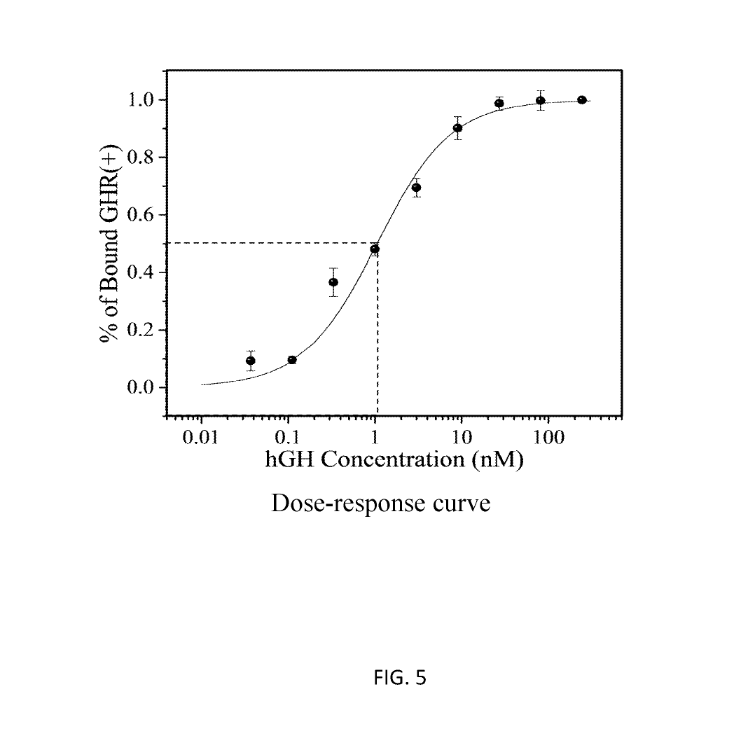

FIG. 4A illustrates the use of a charge polarity transition strategy, using positive molecular charge modulation (“MCM”) and electrokinetic concentration (“EKC”), to determine equilibrium binding parameters for a drug-receptor interaction. At time t, after voltage 405 is applied across the microchannel 425 (and across the cation-selective nanojunction 420) the locations of species are as depicted in a series of figures (a)-(d), corresponding to increasing levels of drug.

As shown in FIG. 4A, when drug is absent, the species that is present is positively charged receptor-peptide pair 440, which is positioned to the right of the nanojunction, as the receptor-peptide pairs having a net positive charge pass the cation-selective nanojunction 420. FIGS. 4B-4F depict the result at increasing concentrations of drug (c_{drug}), resulting in the complexation of an increasing fraction of the receptor-peptide pairs 440 to form receptor-drug complex 450.

FIG. 4E shows images (based on detection of a fluorescence signal from the labeled peptide modulator) across a region of the channels depicted in FIGS. 4A-4D, at a series of drug concentrations 475, ranging from 0 nM to 243 nM. The complex 450 of the drug with the mobility-modified receptor is shown at location 470 along the separation axis for the assay. Location 455 is the ion depletion region. The intensity of the band integrated over the area of the band correlates with the amount of label present, which for band 475, represents the quantity of complex 450. The data can then be represented as standard binding curves from which equilibrium dissociation constants can be determined. FIG. 4E depicts results wherein the drug is human growth hormone (hGH).

FIG. 5 is a semilog plot of the data shown in FIG. 4E. The binding curve shows drug concentration along the x-axis and fractional binding along the y-axis. From the plot, K_{D} can be determined according to well-known relationships, and it can be graphically approximated (see dashed lines) as corresponding to the drug concentration at which a half-maximal response is achieved.

It should be appreciated that kinetic data can also be obtained, for example, based on time-series data of signal accumulation at band 470, which is expected to be sublinear, reflecting a balance between increasing signal intensity with time (as species 450 accumulates), and dissociation of species 450 into free drug and unbound receptor 440, wherein the unbound receptor (owing to charge) is rapidly swept past the nanojunction before appreciable rebinding can occur. In this manner, the off-rate constant for drug-receptor binding can be determined. Given the equilibrium dissociation constant and the off-rate constant, the on-rate constant can be determined as well.

Mobility Enhancement Strategies (e.g., nMCM)

Another example of a molecular charge modulation strategy is a mobility enhancement strategy. Accordingly, a second class of methods disclosed herein relates to enhancing the mobility of at least one of the drug and the receptor, but without tuning the charge on the receptor such that the charge on the modulated receptor-drug complex is opposite in sign to the charge on the free (drug-unbound) modulated receptor. These methods can be referred to as mobility enhancement strategies. It should be appreciated that a desirable direction of mobility enhancement can be either positive or negative, depending on factors including directionality of the electric field.

For example, TPs and TPRs usually have relatively low mobility because most amino acid residues are not electrically charged. On the contrary, oligonucleotides can have high mobility due to high charge-to-mass ratios. By conjugating TPR with an oligonucleotide, the TPR-oligonucleotide conjugate (nTPR) and the TP-nTPR complex could have high mobility at proper degrees of conjugation (DoCs), enabling their separation from the unbound low-mobility TP. Labeling of TP molecules can be performed to visualize the separation; and the method can be used for competitive assays, e.g., where the reference TP molecules are labeled but the test TP molecules are not.

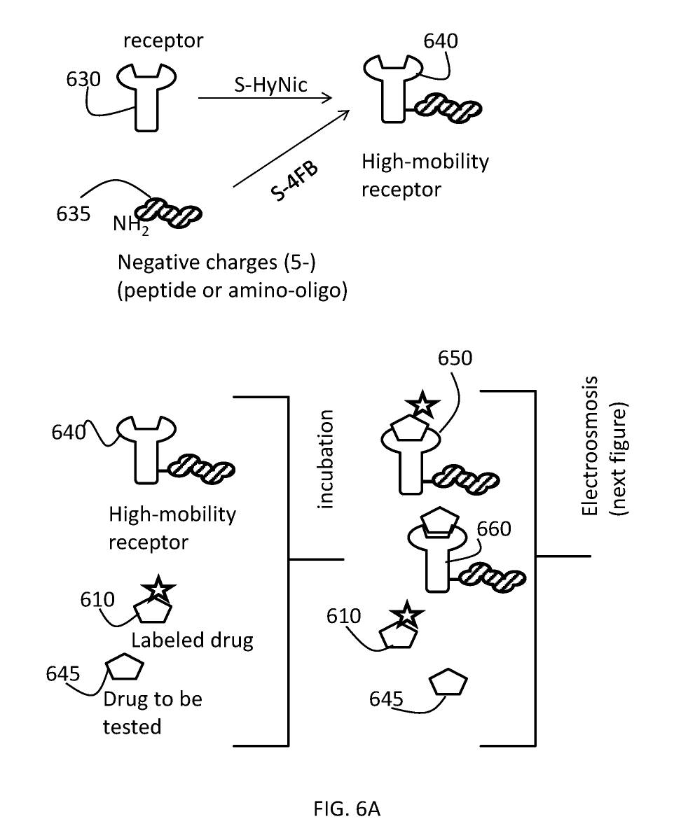

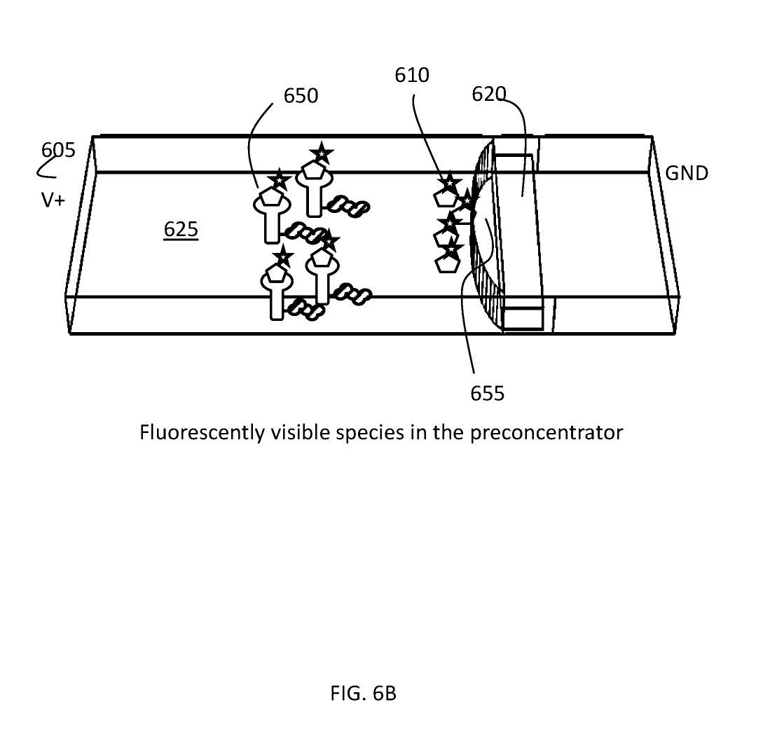

FIG. 6A illustrates a mobility enhancement strategy according to aspects of the present invention. As shown, the mobility of a receptor 630 is increased by increasing net charge on the receptor (by binding to a charged molecule 635) to form high mobility receptor 640. For example, charged molecule 635 can be highly negative, and unlabeled peptides/oligonucleotides, and high-mobility receptor-peptide pairs 640 can have a net negative charge.

As shown in FIG. 6B, the mobility of the modulated receptor 630 is made to be sufficiently high relative to the labeled drug 610 that the mobility of the drug-modulated receptor complex 650 will likewise be appreciably higher than that of labeled drug 610. Complex 650 localizes along the separation axis at a distance from the labeled drug 610, enabling resolution of signal coming from unbound drug 610 and complex 650, and the determination of binding parameters between the labeled drug and the modulated receptor according to well-known pharmacological relationships.

FIG. 6A further illustrates that a mobility enhancement strategy can be combined with a competitive assay. For example, labeled drug 610 can be used as a probe molecule that is displaced by an unlabeled drug to be tested 645, thereby yielding information about the binding properties of drug to be tested 645. As shown, drug to be tested 645 and labeled reference drug 610 both compete for binding to receptor mobility modulated 640. Drug to be tested 645, labeled reference drug 610, and unlabeled high-mobility receptor-peptide pairs 640 can be incubated. In FIG. 6B, the incubated result includes complexes 660 and 650, formed, respectively, with labeled drug 610 and drug to be tested 645. The incubated result (650, 660 (not shown), 645 (not shown) and 610) is inserted into a microchannel 625 of an electrokinetic concentrator and a voltage 605 is applied across the cation-selective nanojunction 620. After a period of time t, an ion depletion area 655 forms on one side of the cation-selection nanojunction 620.

In some embodiments, labeled drug 610 corresponds to an analyte of interest, e.g., it is a labeled version of an analyte drug 645. The binding of unlabeled analyte drug 645 can therefore be visualized by competition experiments, e.g., wherein drug 610 and analyte drug 645 are competitive antagonists for binding at a site on the modulated receptor 640. In some cases, such as where labeled drug 610 is merely a labeled version of analyte drug 645, binding parameters to the modulated receptor will be similar or identical for both drugs 610/645. In other cases, such as where analyte drug 645 differs structurally from labeled drug 610 beyond the addition of a label, binding parameters can be different. Notwithstanding any such differences, competition experiments can be used to infer binding properties for analyte drug 645 based on competition from labeled drug 610, especially in situations where competitive antagonism is established as between the drug species and binding parameters for labeled drug 610 are known.

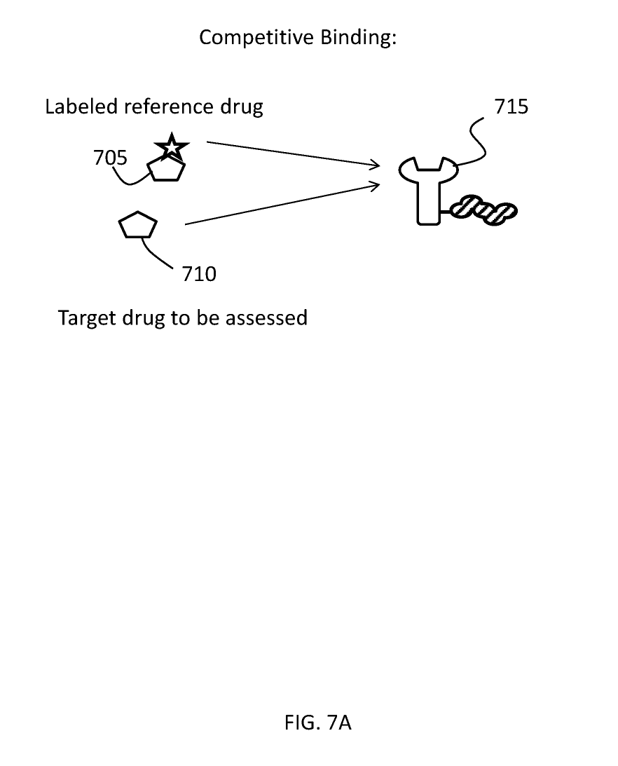

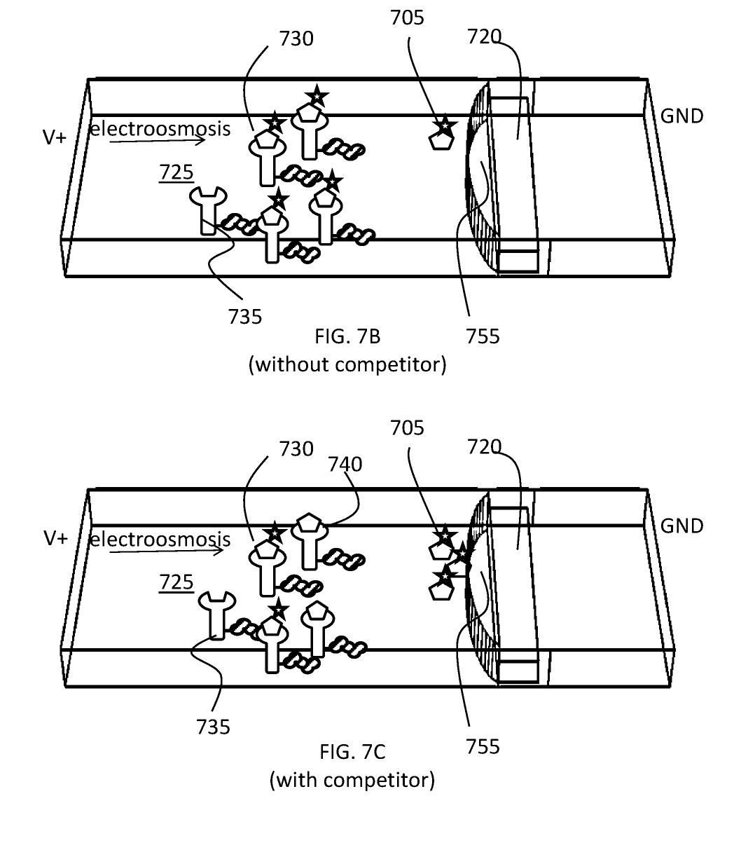

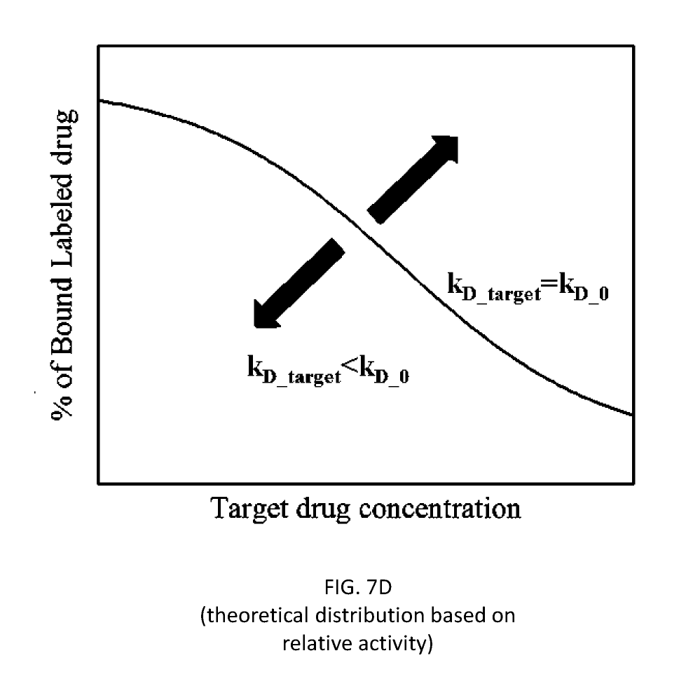

FIGS. 7A-7D further illustrate principles of a competitive assay according to aspects of the present invention.

FIG. 7A depicts competition between a drug to be assessed 710 and a labeled reference drug 705 for binding to a receptor. Highly negative and unlabeled peptides/oligonucleotides are conjugated to the receptor to form unlabeled high-mobility receptor-peptide pairs having a net negative charge 715.

FIG. 7B illustrates the separation and concentration of the labeled reference drug 705 by negative MCM and electrokinetic concentration. Electroosmotic flow in FIG. 7B is from left to right. Negative MCM enables the electrical separation of the receptor-reference drug complex 730 and the unbound reference drug 705. Note that unbound receptor-peptide pairs 735 are depicted slightly upstream of the complex 730, reflecting a slight decrease in mobility of the pairs 735 upon binding to the lower mobility drug 705 to form complex 730. It should be appreciated that if the separation between unbound receptor-peptide pairs 735 and complex 730 is sufficient, then receptor molecules can be labeled (instead of the drugs) and the spatial separation between them exploited to probe drug-receptor interactions.

In FIG. 7C, the competitor drug to be assessed 710 competes with the labeled reference drug 705 for binding to the highly labeled receptor-peptide pairs 740.

FIG. 7D depicts a dose-response curve showing theoretical displacement of labeled drug 730 from modulated receptor 735 by the analyte drug to be assessed 710, assuming that the K_{D} for modulated receptor binding to both drugs are equal. If the K_{D}s are not equal, then the observed curves will be shifted in the directions depicted in the figure. The K_{D} for binding of target drug 710 to modulated receptor can be determined according to standard methods, given, inter alia, the K_{D} for binding of the labeled reference drug 705. The K_{D} for binding of target drug 710 can then be compared with an expected K_{D} for the target drug (e.g., that for a standard drug of known quality).

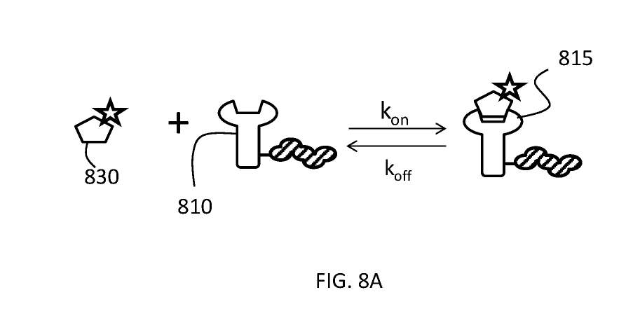

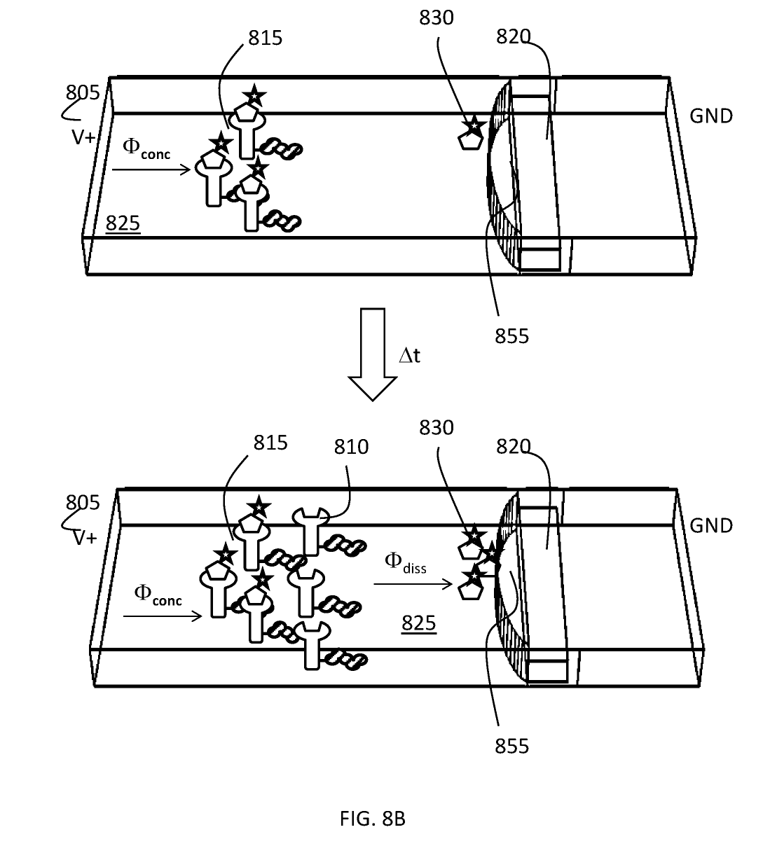

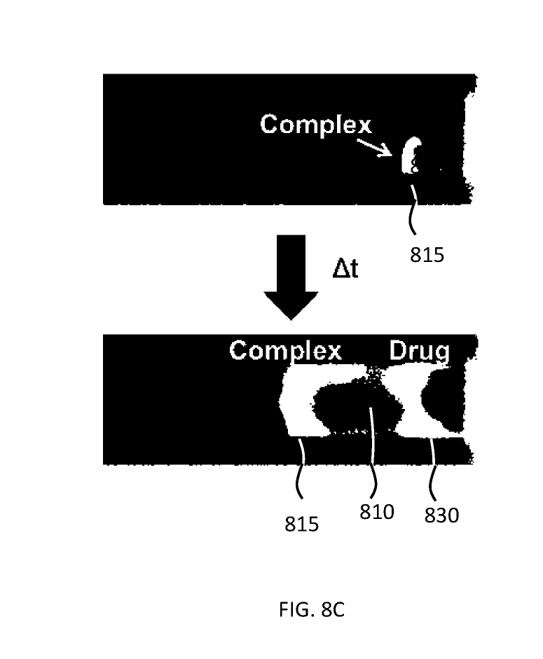

Binding kinetics can also be measured using mobility enhancement strategies. FIG. 8A depicts a fluorescently labeled drug 830 having a binding affinity for a negatively charged receptor-peptide pair 810, forming a labeled negatively charged complex 815, with on and off rate constants as indicated.

FIG. 8B illustrates the measurement of dissociation rate by employing negative MCM and electrokinetic concentration. After voltage 805 is applied across the nanojunction 820 the labeled negatively charged receptor-drug complex 815 is spatially and electrically separated from the unbound labeled drug 830. The complex accumulates at a rate equal to the rate of arrival of complex due to flow minus the rate of dissociation of complex, according to the equations below:

$$\frac{{dn}_{complex}}{dt} = {ϕ}_{conc .} - {ϕ}_{diss .} = {υ}_{EOF} \cdot {c}_{complex \_ bulk} \cdot A - {k}_{off} {n}_{complex}$$

$${n}_{complex} = \frac{{v}_{EOF} {c}_{complex \_ bulk} A}{{k}_{off}} ( 1 - {e}^{- {k}_{off} t} )$$

where n_{complex} is quanity of complex; φ_{conc} and φ_{diss.} are rates of concentration and dissociation, respectively, of complex; v_{EOF} is the velocity of electroosmotic flow; c_{complex}_{_}_{bulk} is the concentration of complex in bulk solution; A is cross sectional area of the channel; and k_{off} is dissociation rate constant.

FIG. 8C shows fluorescence images depicting bands corresponding to complex 815 and drug 830. The expected position of unbound receptor (not containing label, and therefore not visible) is shown for reference at 810. The gain in intensity of band 815 can be used with standard curve fitting techniques to determine k_{off} according to the above integrated form of the differential equation. It should be appreciated that kinetic data can also be garnered from the gain in signal intensity of band 830 as a function of time.

### Example I: Measurement of Binding Affinity of hGH (Human Growth Hormone) to hGH-Receptor

The binding affinity of hGH to hGH-receptor, reported as a K_{D}, was determined by two formats of assays: a direct assay by charge polarity transition (by positive MCM) and a competitive assay by mobility enhancement (negative MCM).

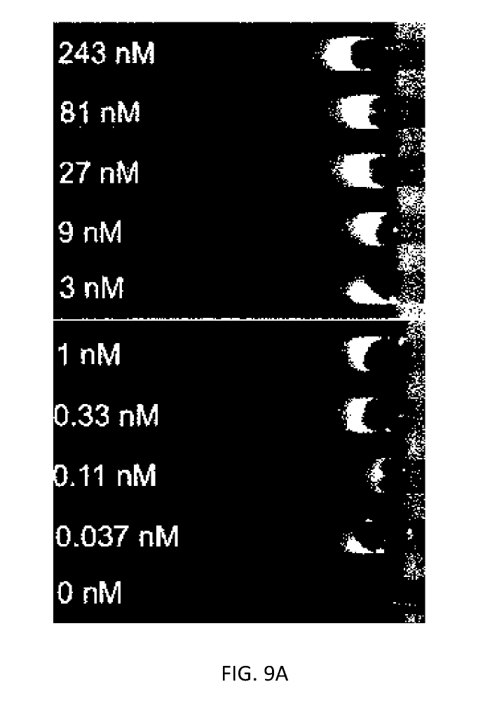

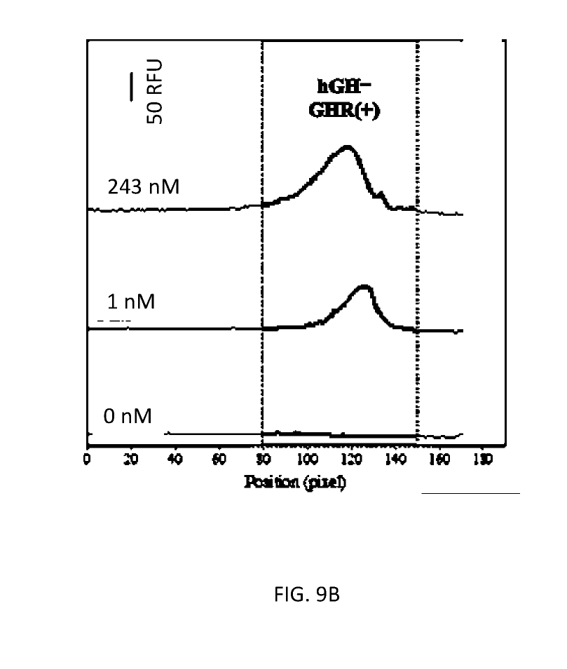

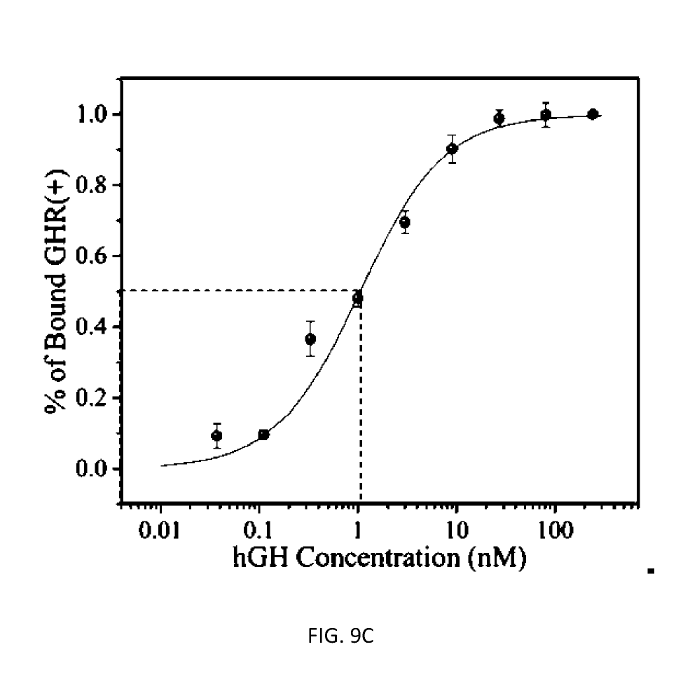

For the direct assay, 10 nM positively modulated hGH receptor (GHR(+)) and 0-243 nM hGH were incubated 30 min before testing. While the positively modulated hGH receptor, GHR(+), was not concentrated, the complex GHR(+)-hGH was negatively charged and concentrated, with stronger intensity at higher hGH concentrations. The dose-response curve was plotted based on the fluorescence intensity of the concentrated complex. The k_{D} measured by this assay was ˜1 nM. FIG. 9A-9C shows the experimental results of the direct assay by positive MCM for hGH. FIG. 9A shows fluorescence images of concentrated hGH-GHR(+). FIG. 9(b) shows fluorescence profiles of concentrated plugs. FIG. 9C shows the dose-response curve for hGH.

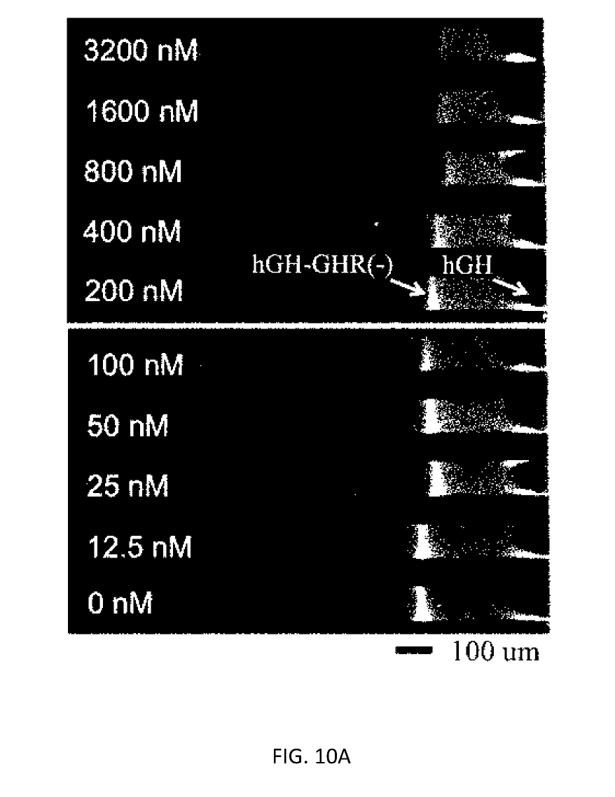

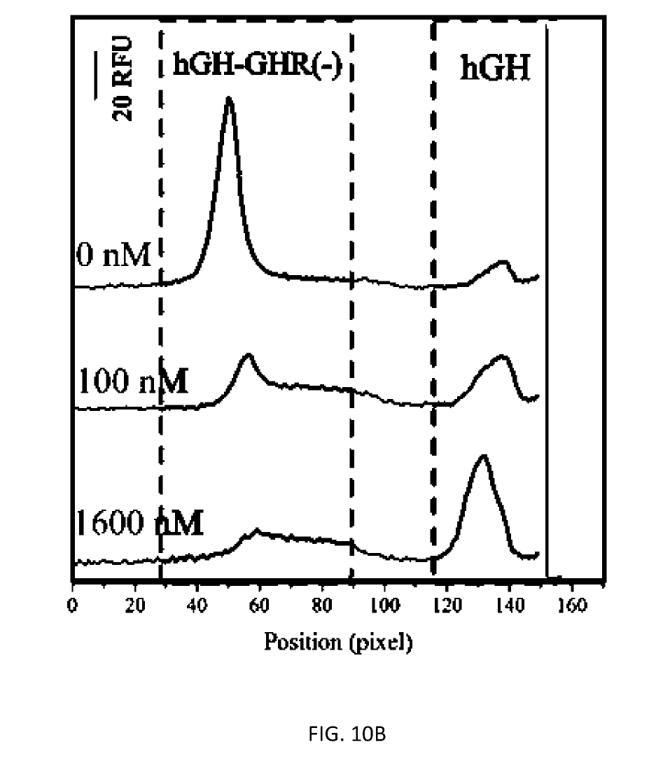

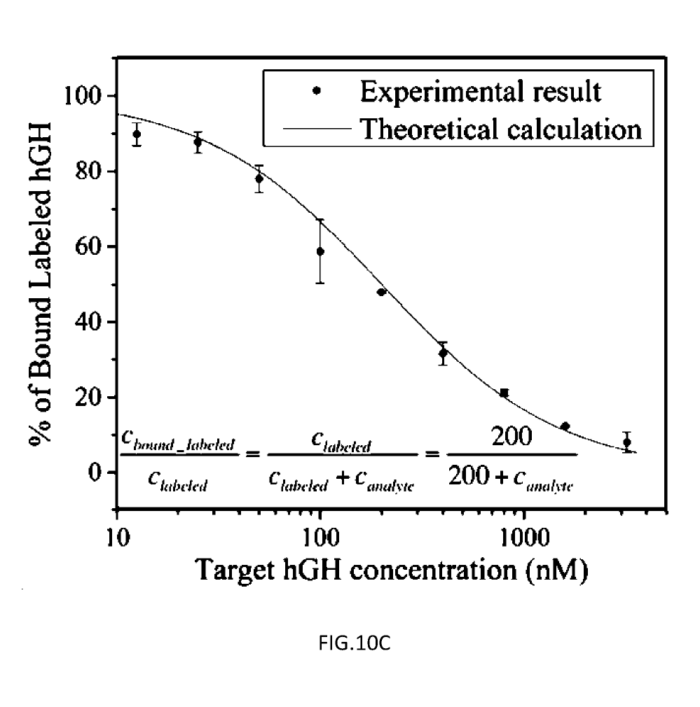

For the competitive assay by negative MCM, 200 nM of negatively modulated hGH receptor (GHR(−)), 200 nM of labeled hGH and 0-3200 nM of target hGH were incubated for thirty minutes. The buffer is 0.1×PBS. A voltage of 30 V was applied for testing. FIG. 10 shows the results of the competitive assay for hGH. FIG. 10A shows the fluorescence images of concentrated hGH-GHR(−) complex and free labeled hGH. FIG. 10B shows fluorescence profiles showing different ratios of bound and free labeled hGH due to competitive binding, at concentrations of 0 nM, 100 nM, and 1600 nM unlabeled hGH. FIG. 10C depicts the displacement of bound labeled hGH by unlabeled hGH. The negatively modulated hGH receptor, GHR(−), had greatly enhanced mobility, which enabled the separation of reference labeled hGH* and GHR(−)-hGH* complex. After adding target hGH as a competititor, the peak of GHR(−)-hGH* complex decreased due to competitive binding. The dose-response curve in FIG. 10B shows that all of the resulting experimental data falls on the line, which was calculated under the assumption that the target hGH had the same activity as the reference. This suggests that the target hGH had the same affinity as the reference hGH.

### Example IA: Measurement of Binding Affinity of Other Substances to Other Receptors

In direct assays, incubation mixtures of 0 nM to 270 nM interferon alpha 2b (IFNα2b) and 5 nM positively-modulated interferon alpha 2b receptor (IFNR) (pIFNR), and 0 nM to 81 nM (G-CSF) granulocyte colony stimulating factor (G-CSF) and 1 nM positively modulated granulocyte colony stimulating factor receptor (GCSFR) (pGCSFR) in 0.1×PBS were tested in the EC device. After 30 min incubation, the mixtures were tested in EC devices at 30 V for 15 min. The dose-response curves were plotted by fitting the experimental results with the classic second-order binding model.

In competitive assays, mixtures of 0-3200 nM test TPs, 200 nM reference TPs, 200 nM nTPRs in 0.1×PBS were incubated for 30 min and concentrated for <1 min.

### Examples I and IA: Further Information

In direct assays, without adding hGH, IFNα2b, and G-CSF, no concentrated band was observed in the electrokinetic concentration (EC) device, as expected for only positively charged receptors pGHR, pIFNR, and pGCSFR being present in the samples. With the test TPs, the negatively charged hGH-pGHR, IFN-pIFNR, and GCSF-pGCSFR were formed and generated concentration bands in the EC device, which became stronger at higher concentrations of TPs. As the dose-response curves indicated, hGH (K_{D}=˜1 nM) and G-CSF (K_{D}=˜0.7 nM) had much higher affinity for their receptors than IFNα2b (K_{D}=˜15 nM), in agreement with literature results and those obtained from other methods.

In competitive assays, test samples of TPs (unlabeled) (hGH, IFNα2b, and G-CSF) competed with corresponding labeled (Alexa Fluor 488) reference samples for binding to nTPRs. The percentage of reference TPs in bound state decreased as more test TP molecules were added, leading to a decrease in the band of the (reference TP)—nTPR complexes and an increase in the band of unbound reference TPs. The experiment results indicated the test TP samples had the same K_{D}s as their references. Meanwhile, the dose-response curve shifted right when the test sample had a higher K_{D} and left when lower, which could be used as a criterion of assessing the K_{D} of a TP sample. The equal-K_{D} curve denoted the dose-response curve of the case where the test and reference TP had the same K_{D}. The Equal-K_{D} curves of hGH and G-CSF overlapped, because the binding efficiency is not sensitive to K_{D} when the TP and TPR concentrations are much higher than K_{D}. For each TP, the assays were conducted in two 5-channel devices, which enabled measurement of 10 concentrations.

### Example II: Measurement of Binding Affinity of IFN (Interferon Alpha 2b) to IFN-Receptor

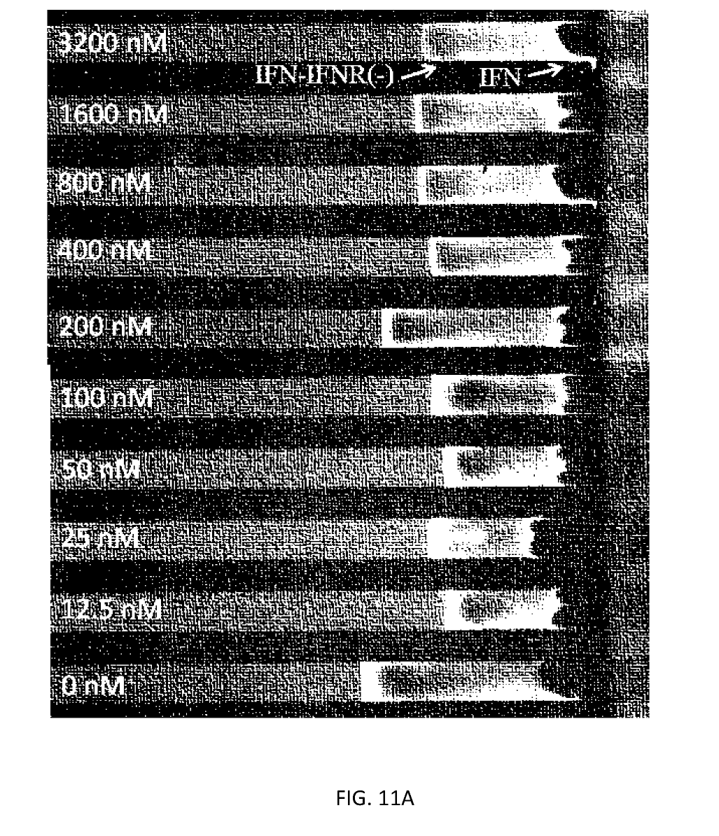

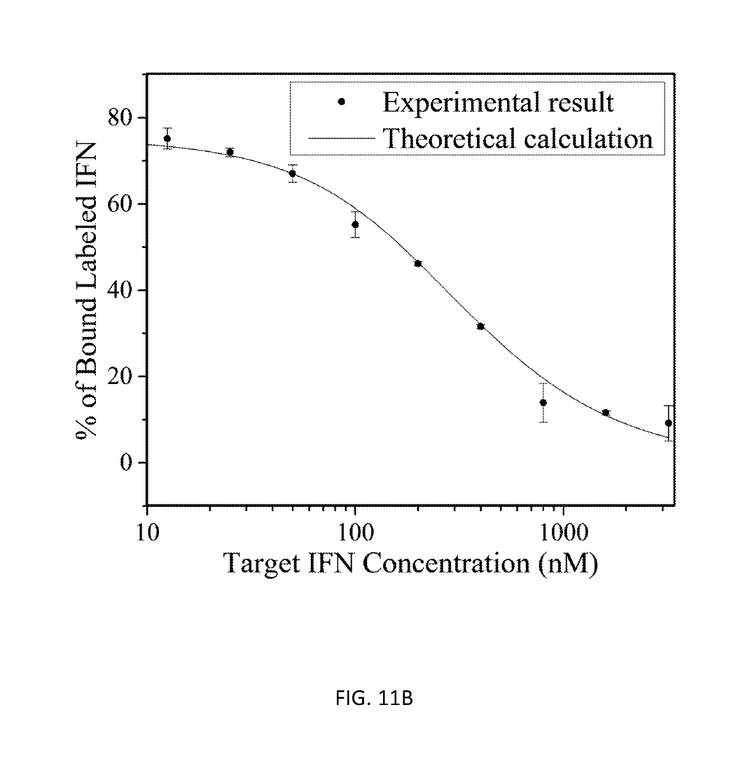

FIGS. 11A-11B show the experimental results of the competitive assay by negative MCM for IFN. FIG. 11A shows the fluorescence images of concentrated IFN-IFNR(−) complex and free labeled IFN. FIG. 11B shows the corresponding dose-response curve.

### Example III: Measurement of Binding Affinity of GCSF (Granulocyte Colony Stimulating Factor) to GCSF-Receptor

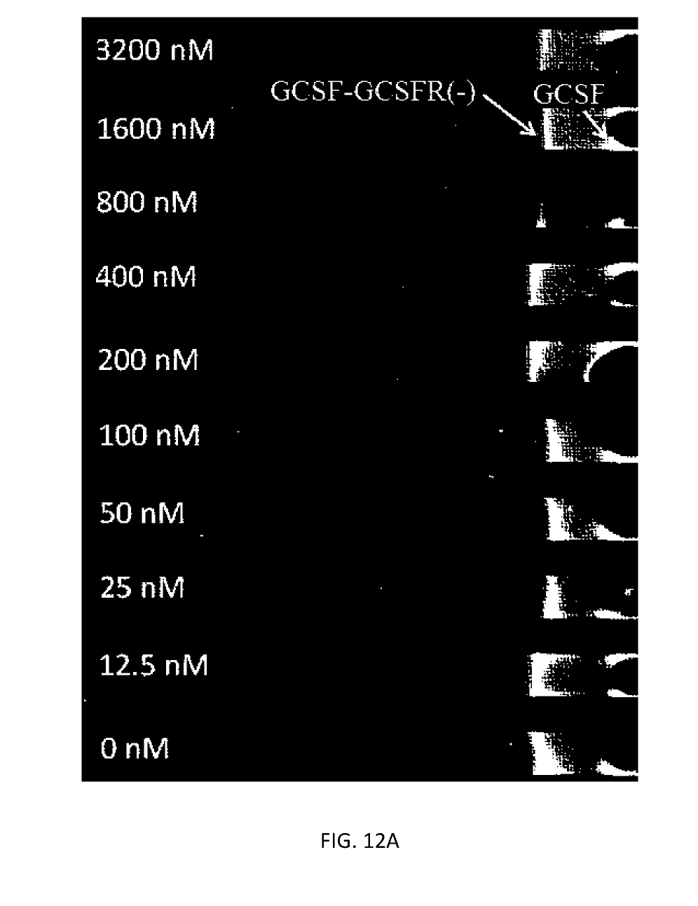

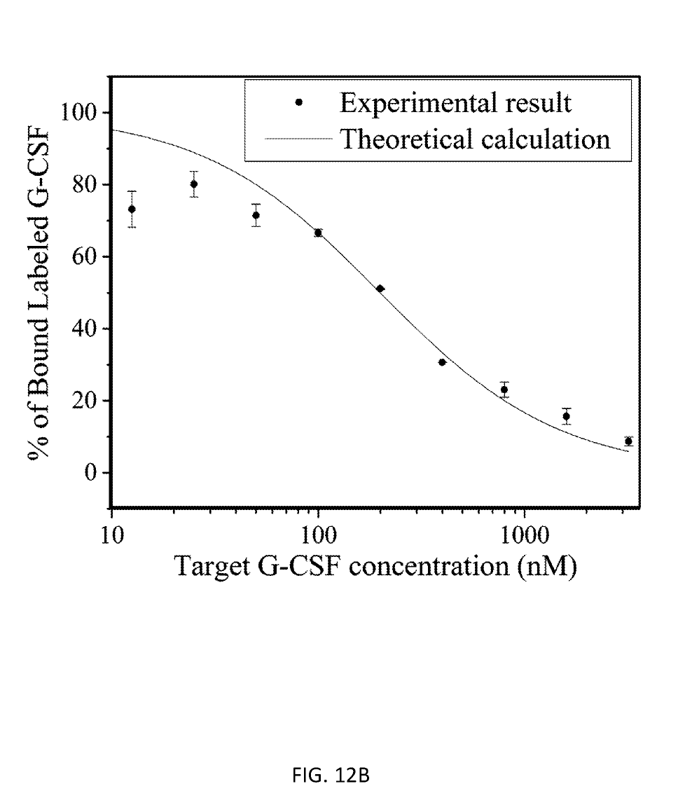

FIGS. 12A-12B show the experimental results of the competitive assay by negative MCM for GCSF. FIG. 12A shows the fluorescence images of concentrated GCSF-GCSFR(−) complex and free labeled GCSF. FIG. 12B shows the corresponding dose-response curve.

### Example IV: Measurement of Binding Kinetics

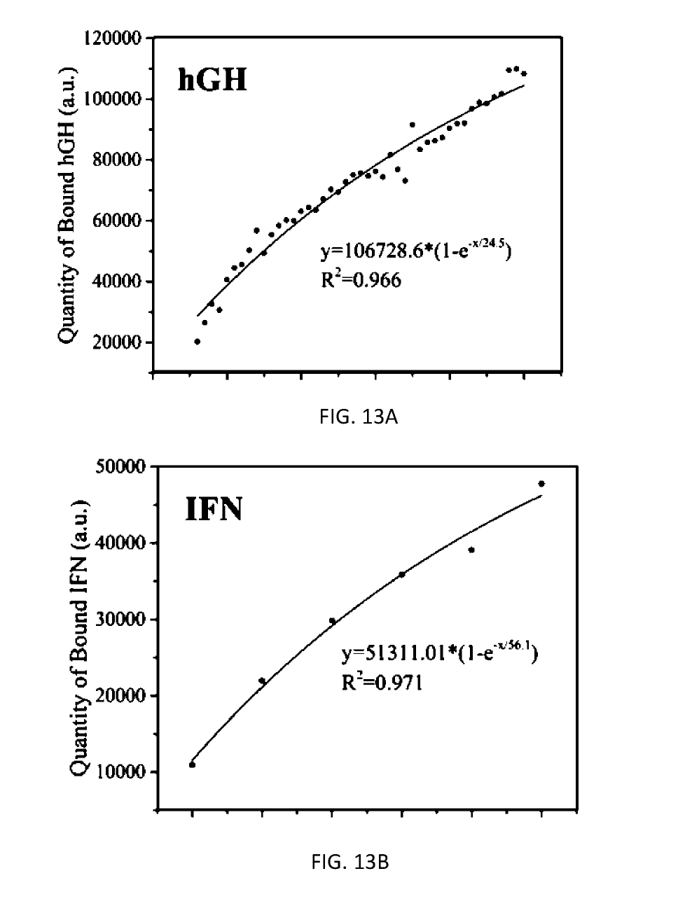

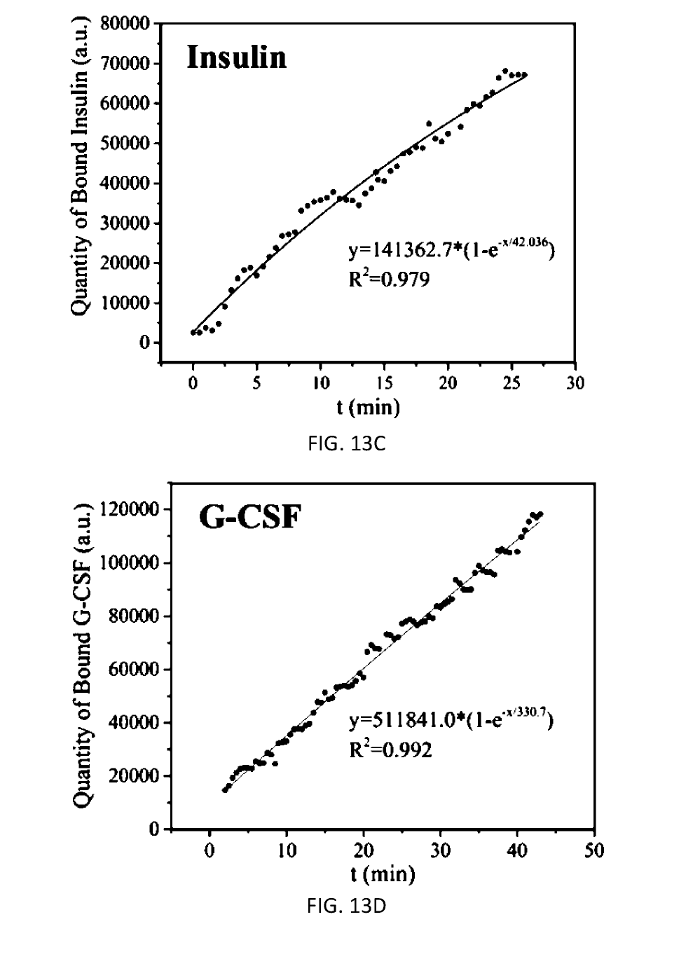

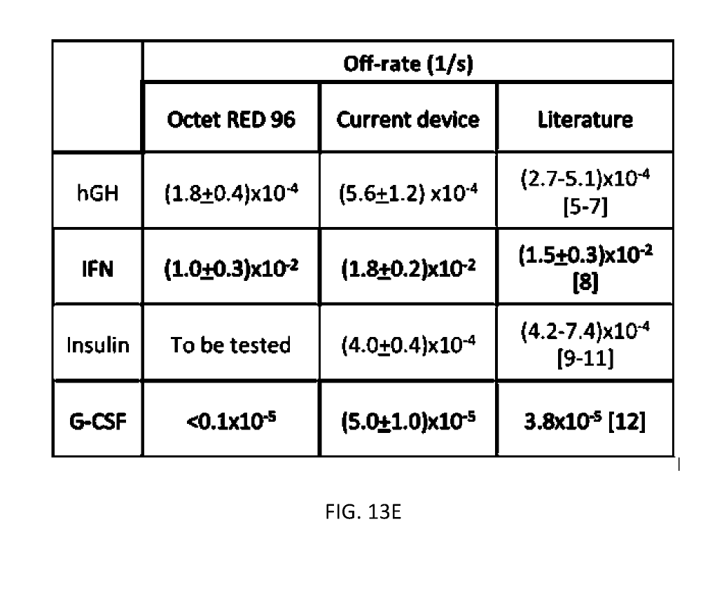

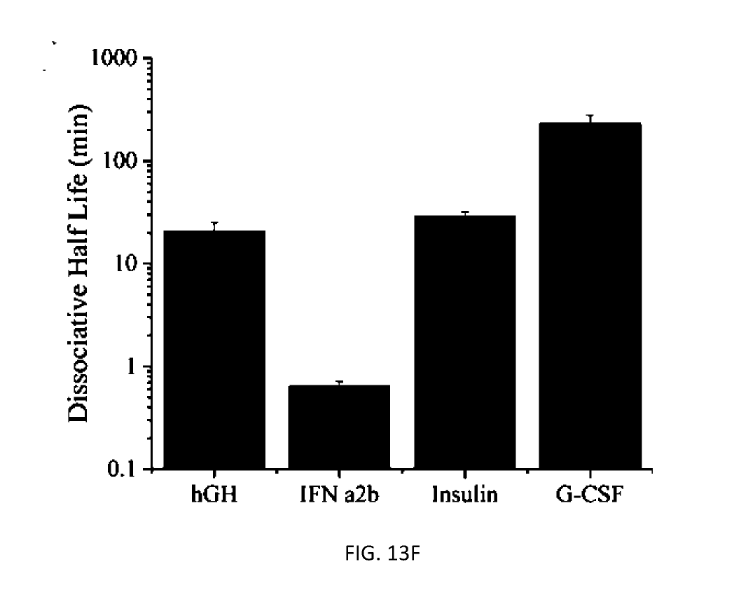

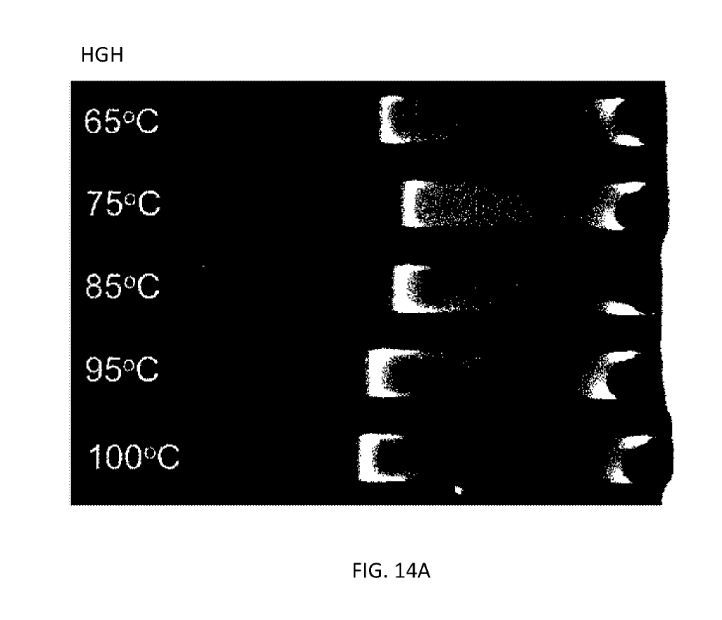

The dissociation rate of drug-receptor binding, k_{off} was measured by monitoring the evolution of the peak of complex and fitting it with the aforementioned derived equation. FIG. 13 shows the results of the binding kinetics measurements. FIGS. 13A-13D shows the evolution of the peak of receptor-drug complex for hGH, IFN, G-CSF and Insulin. The results shown in FIGS. 13A-13D indicate that the four biologics studied (hGH/IFN/G-CSF/Insulin) had dramatically different dissociation rates and dissociative half-lives. The dissociative half-life of IFN was less than 1 min, but that of G-CSF is as long as 330 min, indicating their different functioning dynamics in human body. FIG. 13E shows the comparison of off-rates measured by different methods. FIG. 13E indicates the hGH and GHR binding has an off-rate of (5.6±1.2)×10⁻⁴ l/s, which conforms well with the results from literature and commercial equipment (Octet RED 96, Fortebio). The off-rate indicates that hGH-GHR binding has a dissociative half-life of 20.6±4.4 min. FIG. 13F shows the dissociative half-lives of different biologics—hGH, IFN, G-CSF and Insulin.

### Example V: Stability Assessment

As background, during the production, transport and storage, biologics may be exposed to stress conditions which could degrade the biologics. Therefore, it can be important to assure the safety of biologics before they are administered in patients. A quick way to determine whether a biologic exposed to stress conditions has degraded is by a competitive assay by negative MCM. The K_{D} of the biologic exposed to stress and the K_{D} of the labeled reference drug are measured. If the K_{D} of the biologic exposed to stress conditions is equal to the K_{D} of the reference drug then this indicates that 50% of the reference biologic is bound to the receptor and 50% of the biologic exposed to stress is bound to the receptor. If the K_{D} of the biologic exposed to stress is greater than the K_{D} of the reference biologic, then this indicates that the over 50% of the reference biologic is bound to the receptor while less than 50% of the biologic exposed to stress is bound to the receptor.

Restated, TPs can be exposed to various stress factors during production, storage and final administration, which may degrade TPs and cause loss of efficacy or even arouse severe immune responses.

Degradation of TPs was determined in a competitive assay where the concentrations of the test TP, labeled standard TP, and nTPR were 1:1:1. For intact test TP, 50% of standard TP was bound, while for degraded test TP, >50% of standard TP was bound due to reduced activity of test TP, based on which the relative activity of test TP was rapidly measured. Degradation of hGH and G-CSF was compared under elevated temperature, light exposure, oxidative stress, and after long-term storage according to the ICH Q1A and ICH Q5C guidelines.

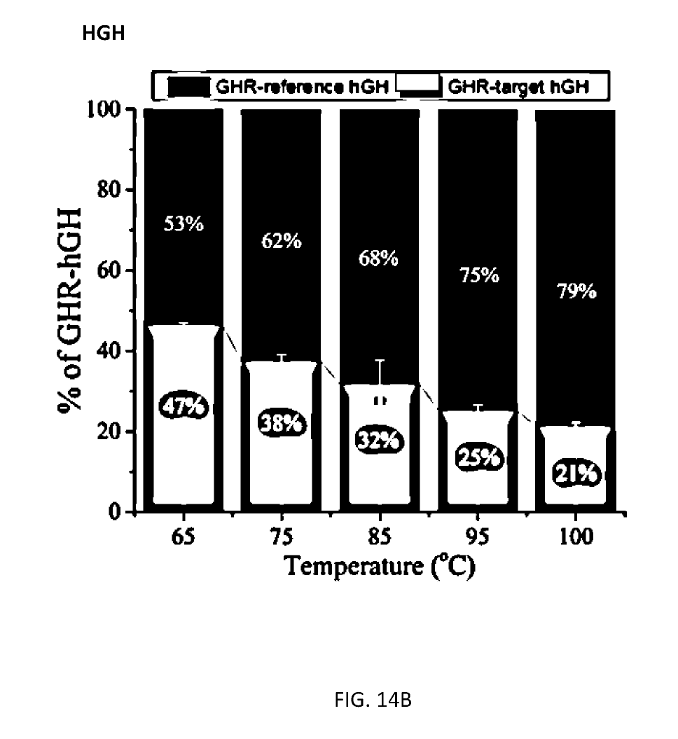

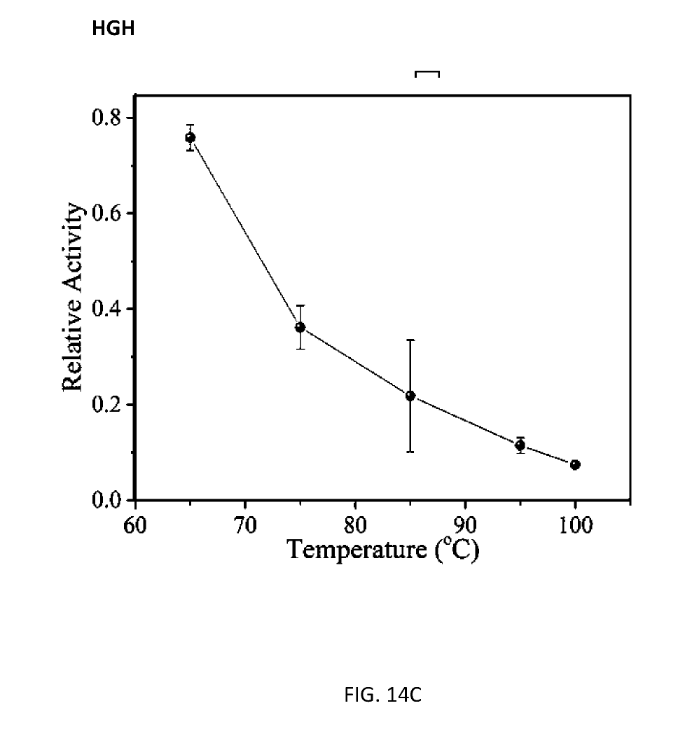

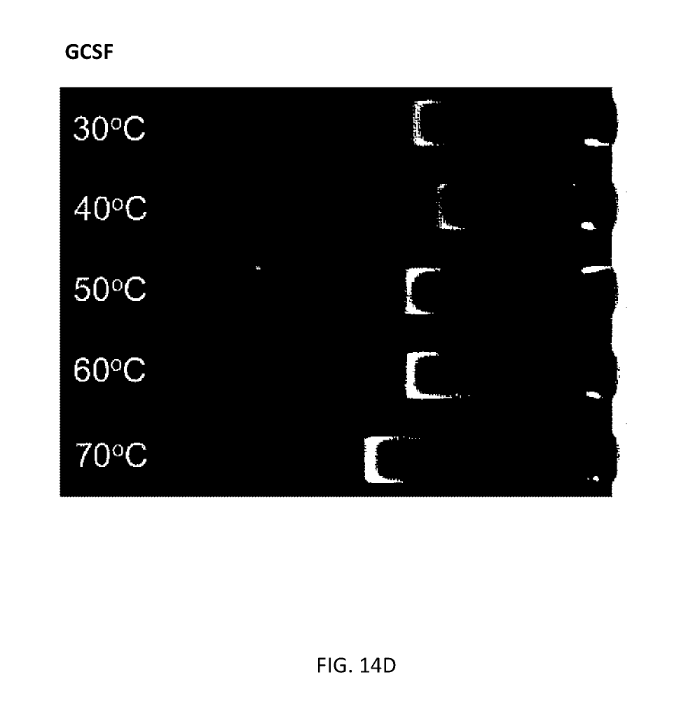

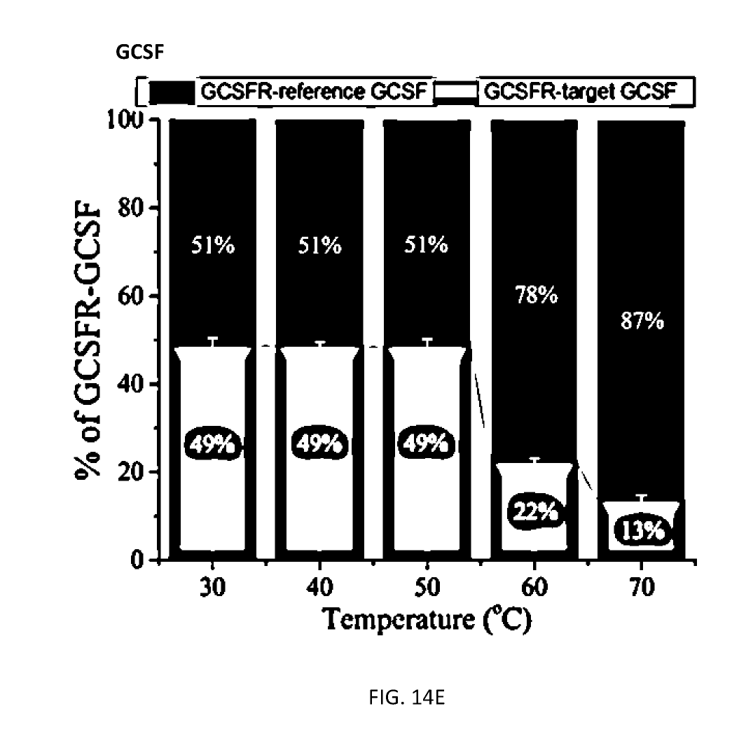

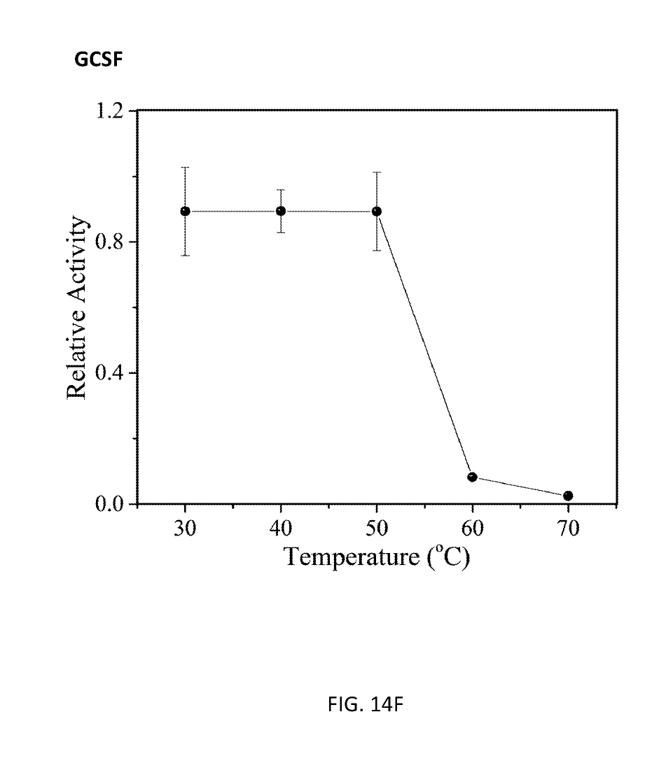

As an example of TP degradation determination using the nMCM-EC platform, the degradation of thermally treated hGH was tested. FIGS. 14A-14C show that the activity of hGH was not significantly lost at 65° C., but above 75° C., its activity dropped below 40%.

As the treatment temperature increased, the intensity of the band of (reference hGH)-nGHR complex also increased, indicating that thermally treated hGH became less active at higher temperatures. Based on the result in FIG. 14A, the percentage of nGHR bound to reference hGH and to test hGH was calculated and shown in FIG. 14B. While almost 50% of nGHR bound to target hGH at 65° C., this percentage decreased at higher temperatures and dropped to only 21% at 100° C. Thermally treated hGH almost kept its full activity at 65° C., but showed dramatic loss of activity from 75° C. (FIG. 14(C).

In comparison, G-CSF was more thermally unstable, which experienced almost complete loss of activity between 50° C. and 60° C. FIGS. 14D-14F show results with G-CSF after exposure to thermal stress.

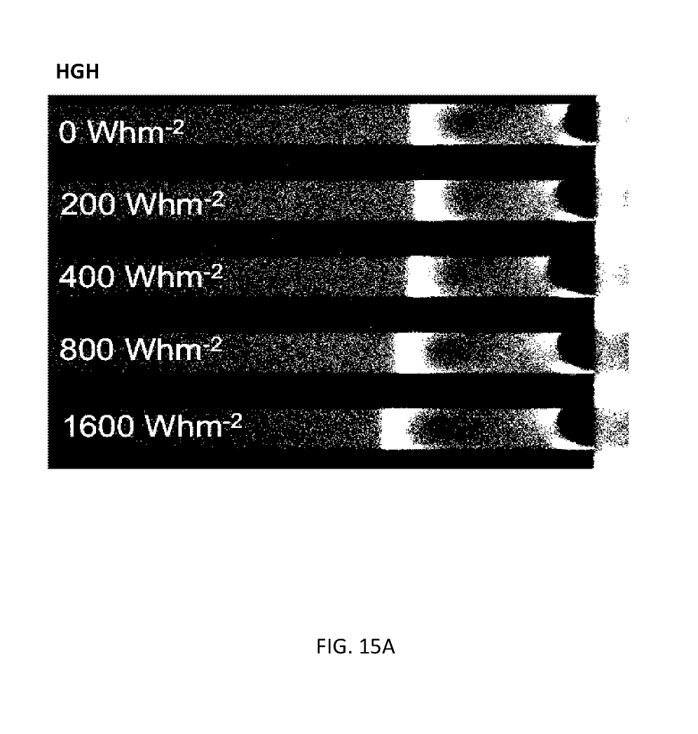

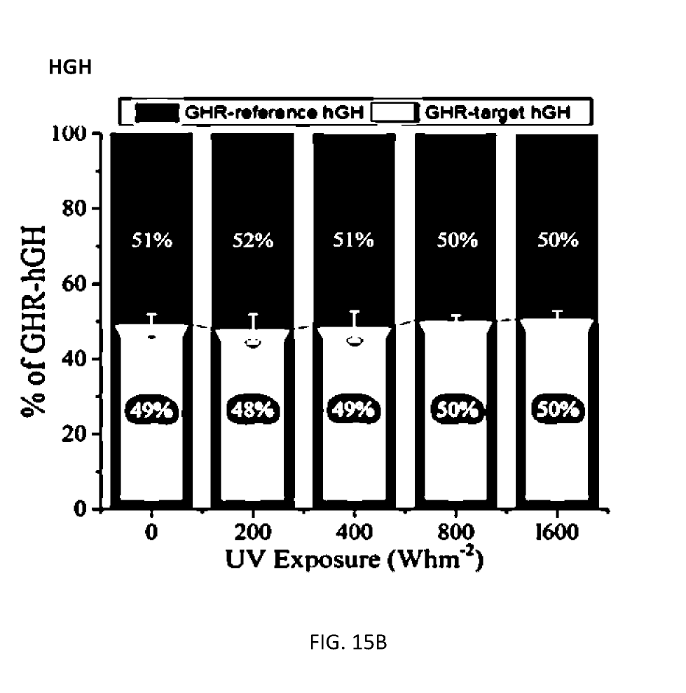

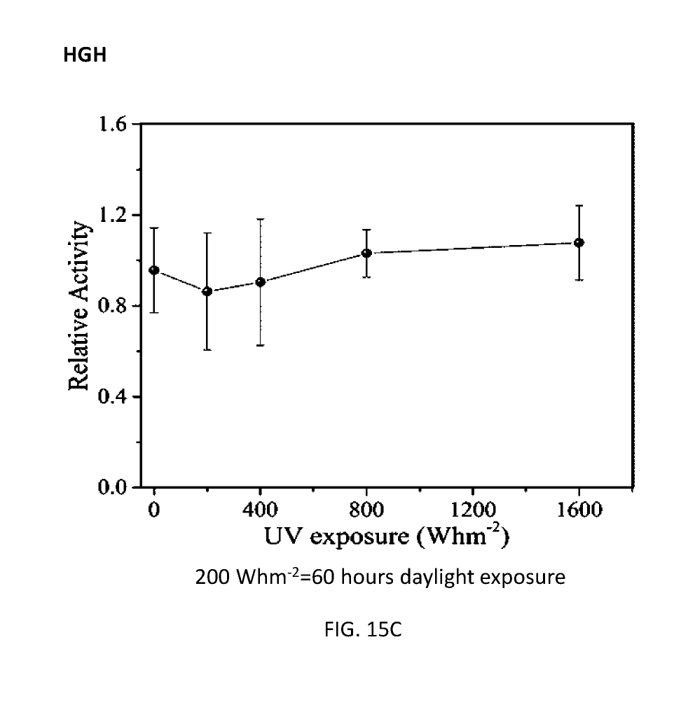

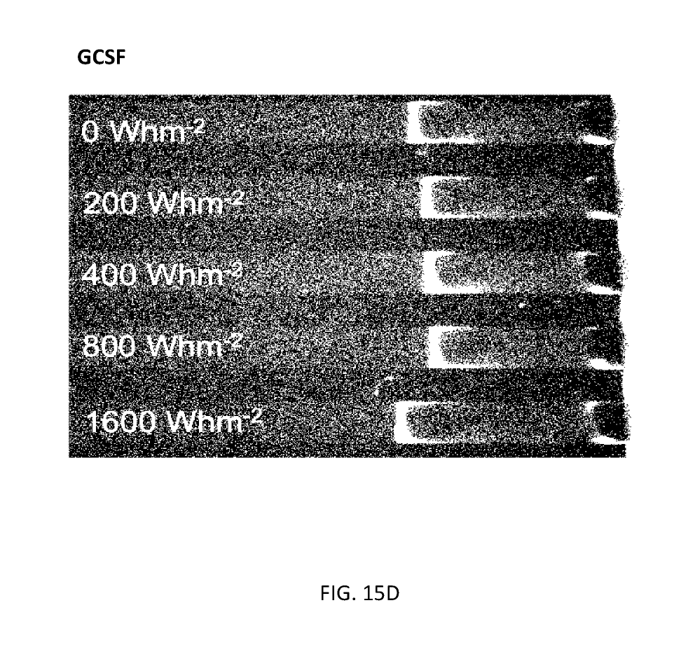

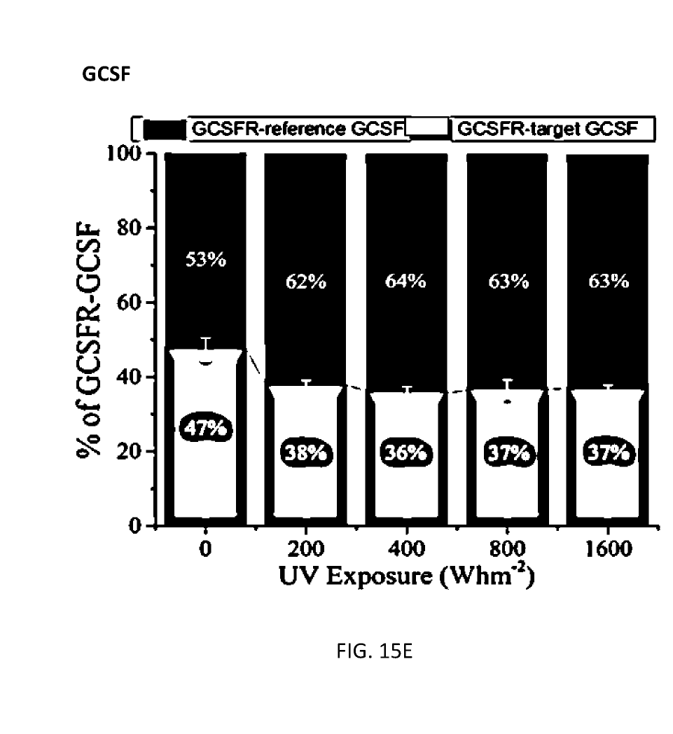

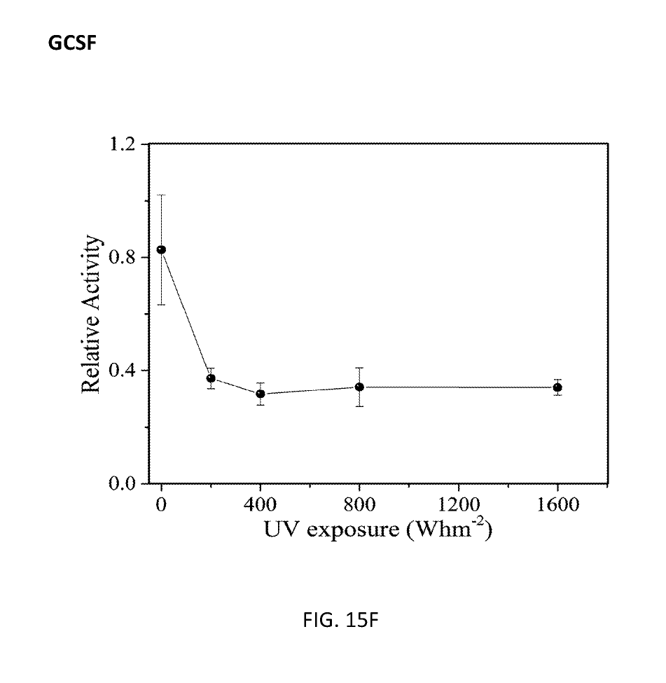

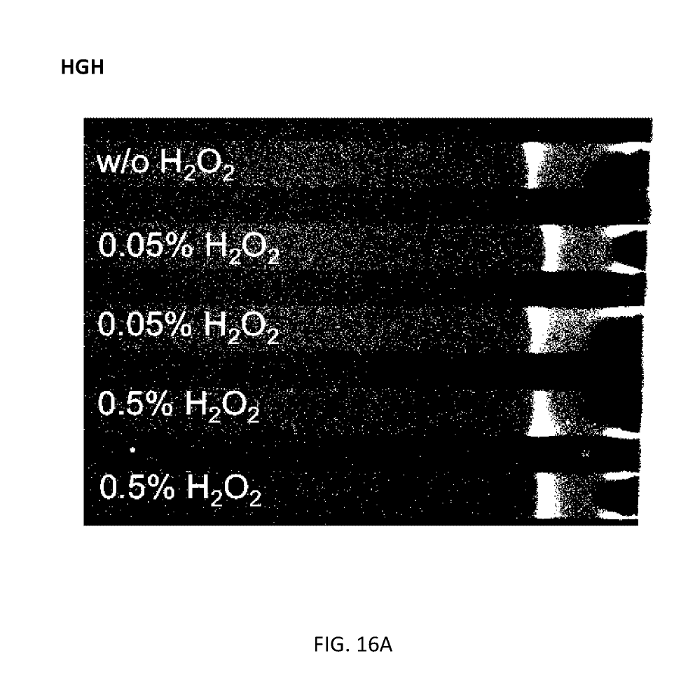

Results of UV exposure, oxidation and long-term storage also suggested that G-CSF was much more susceptible to stresses than hGH (FIGS. 15, 16, and 17) This facile and rapid (30+1 min) assay well suits for rapid on-line quality testing of TPs produced at PoC (point-of-care).

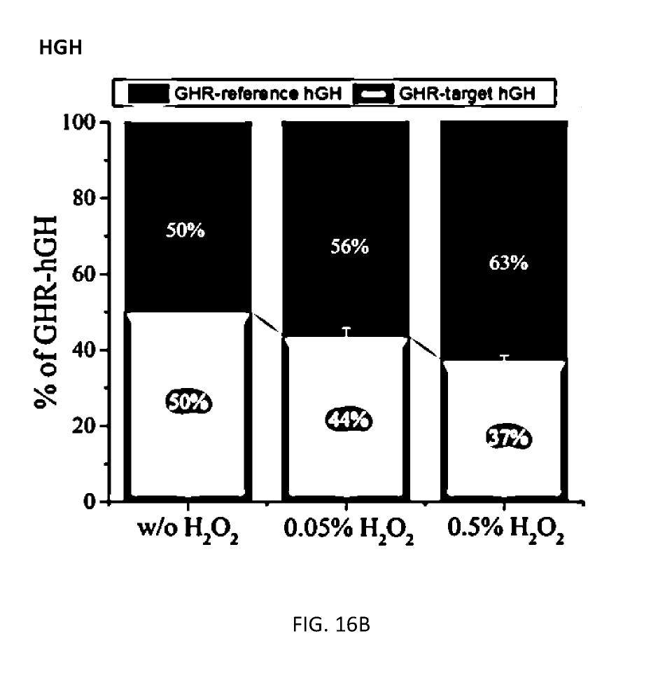

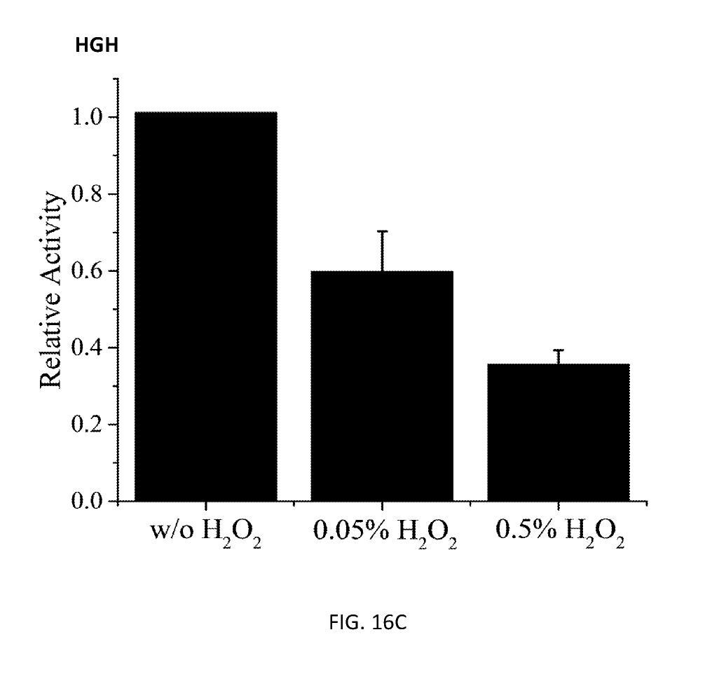

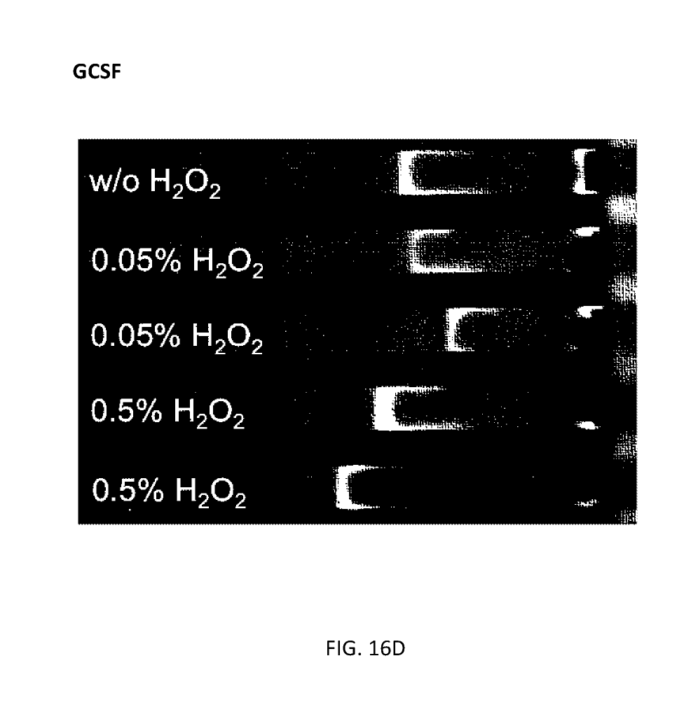

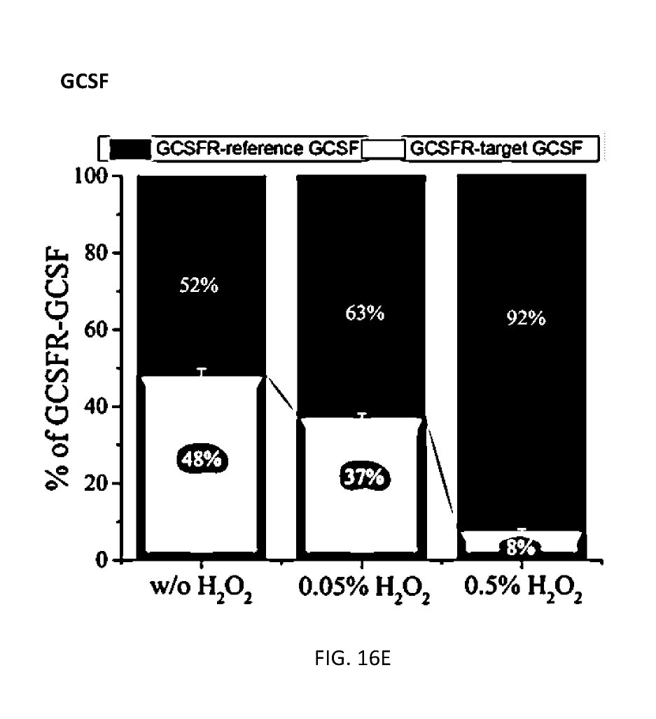

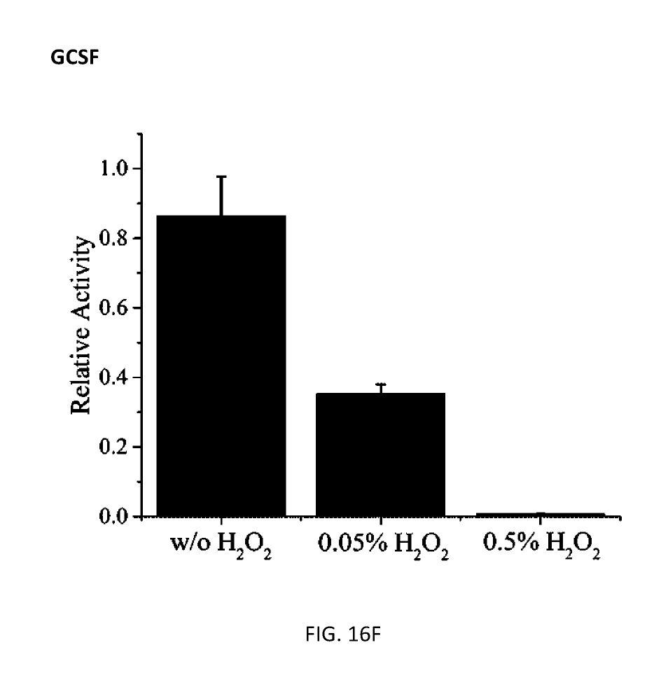

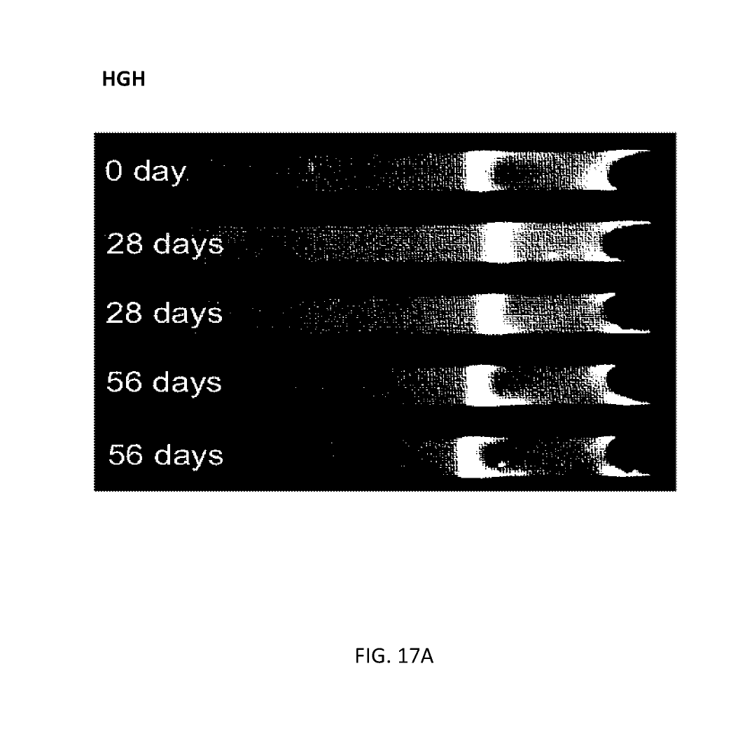

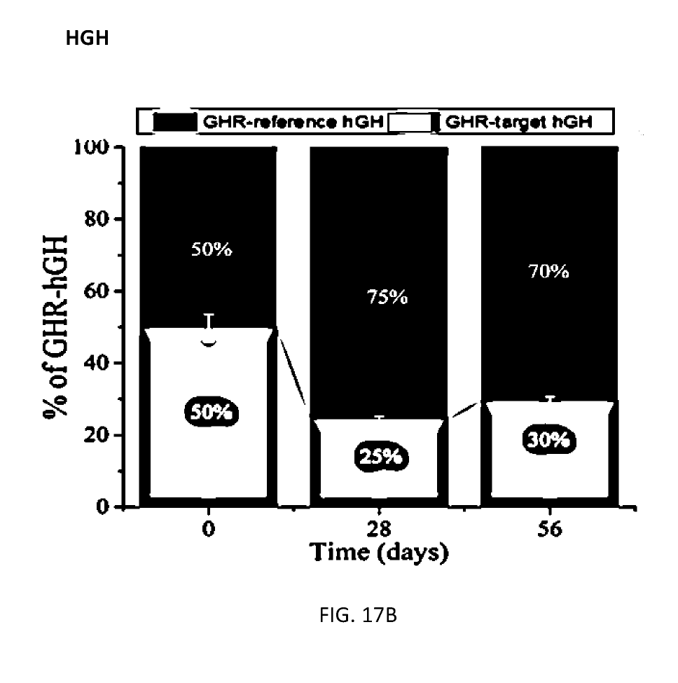

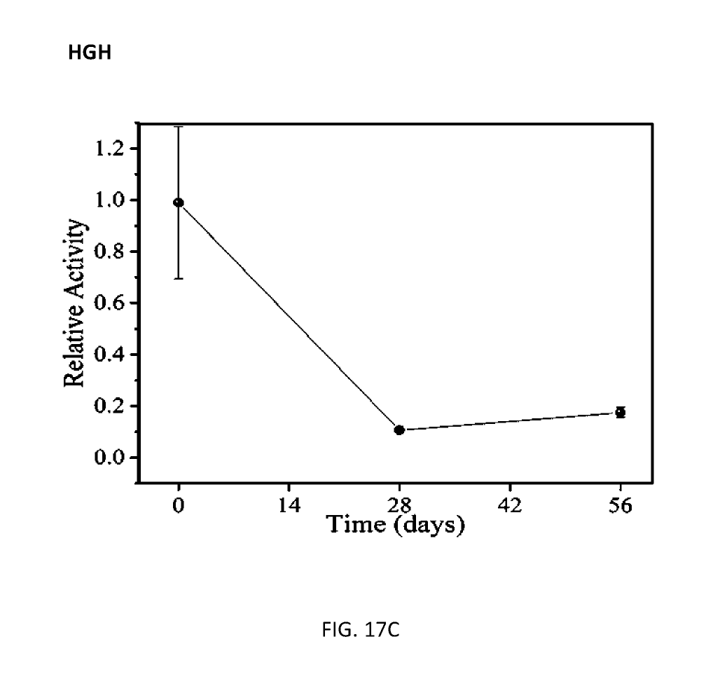

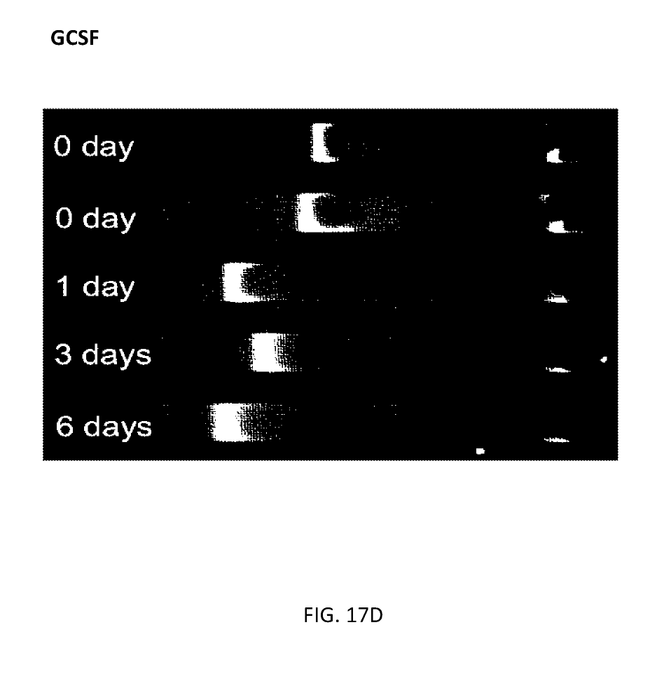

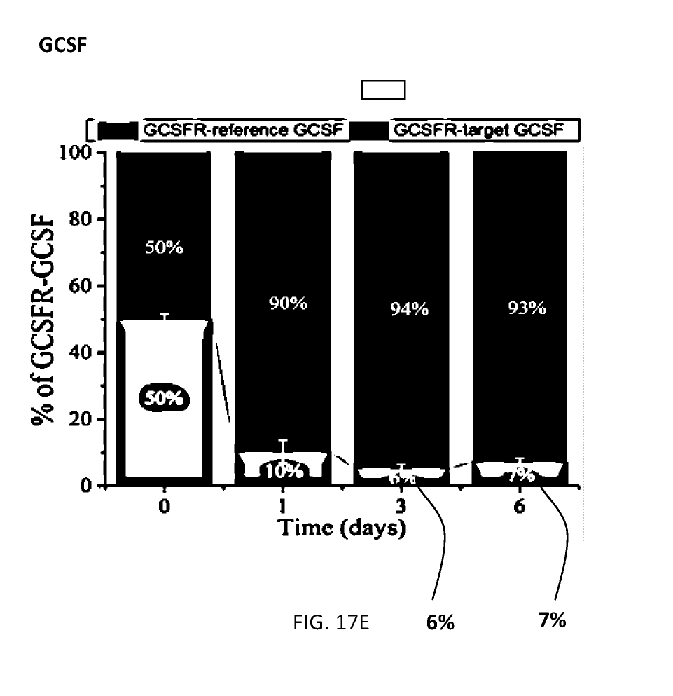

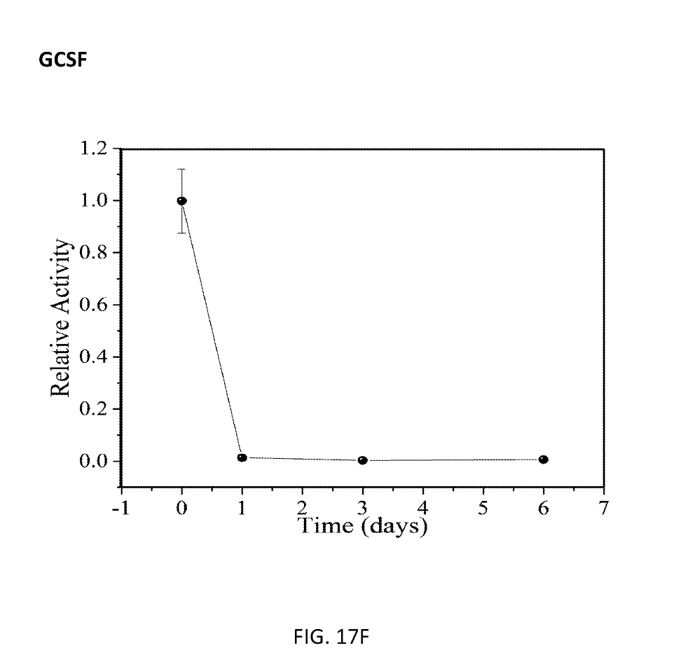

FIGS. 15A-15F show the results of evaluating hGH and G-CSF after exposure to light. FIGS. 15A-15C show that hGH did not sure significant loss activity after being treated with UV exposure up to 1600 Whm⁻². FIGS. 16A-16F show the results of evaluating hGH and G-CSF after exposure to oxidative stress. FIGS. 16A-16C shows that the activity of hGH dropped to −60% after being treated with 0.05% H₂O₂, and ˜35% after being treated with 0.5% H₂O₂, indicating that hGH was susceptible to oxidative stress. FIGS. 17A-17F show the results of evaluating hGH and G-CSF for long-term stability. FIGS. 17A-17C show that the after being incubated at 37° C. for 4 weeks, the activity of hGH dropped to 10%.

### Example VI

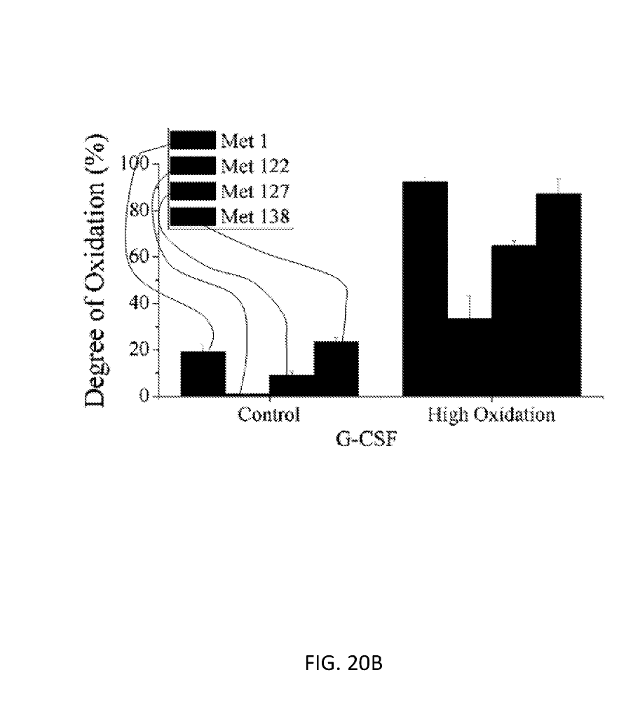

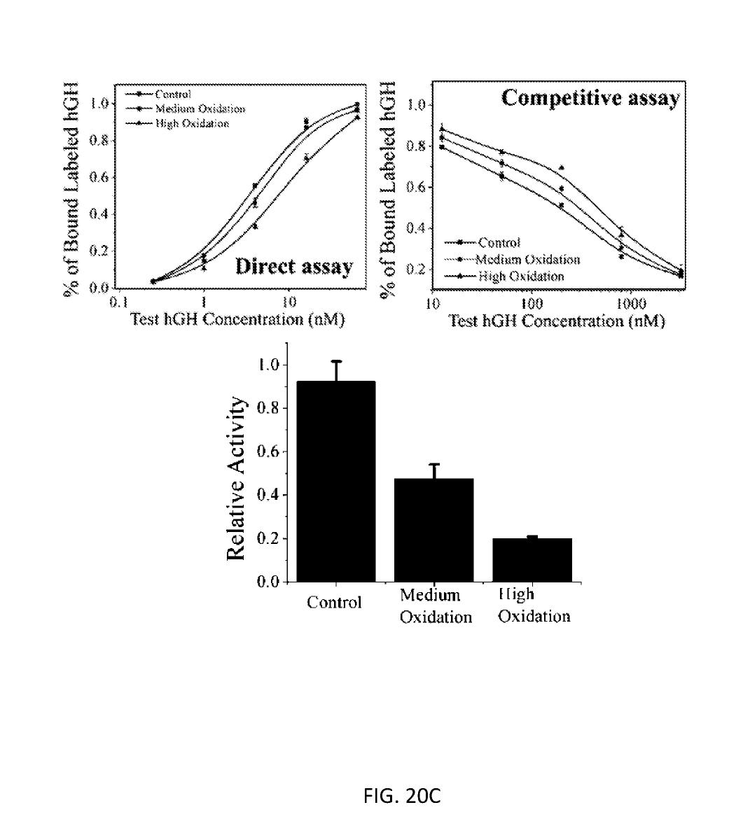

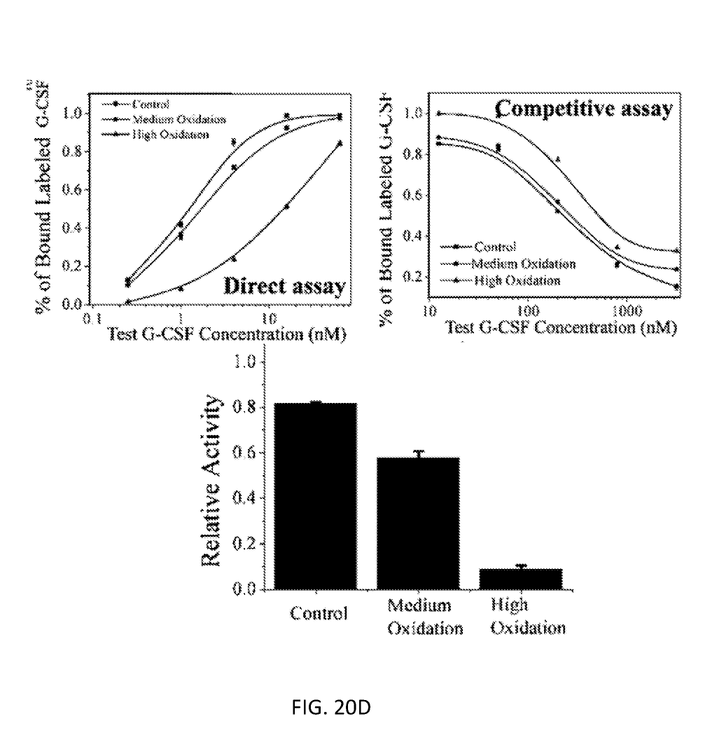

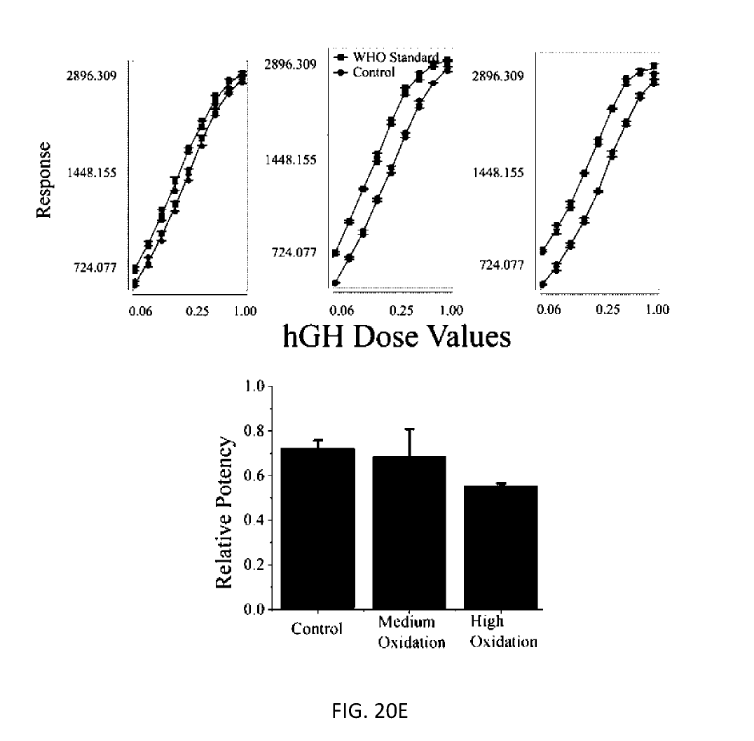

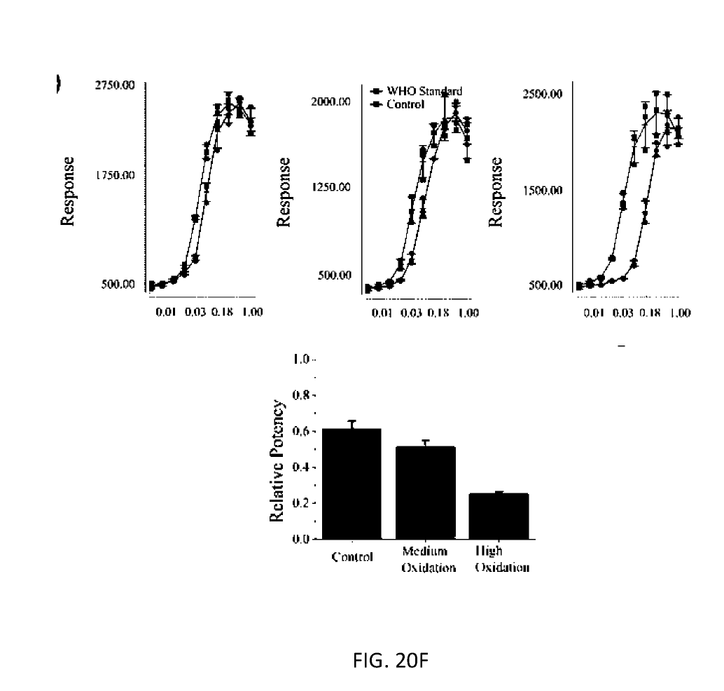

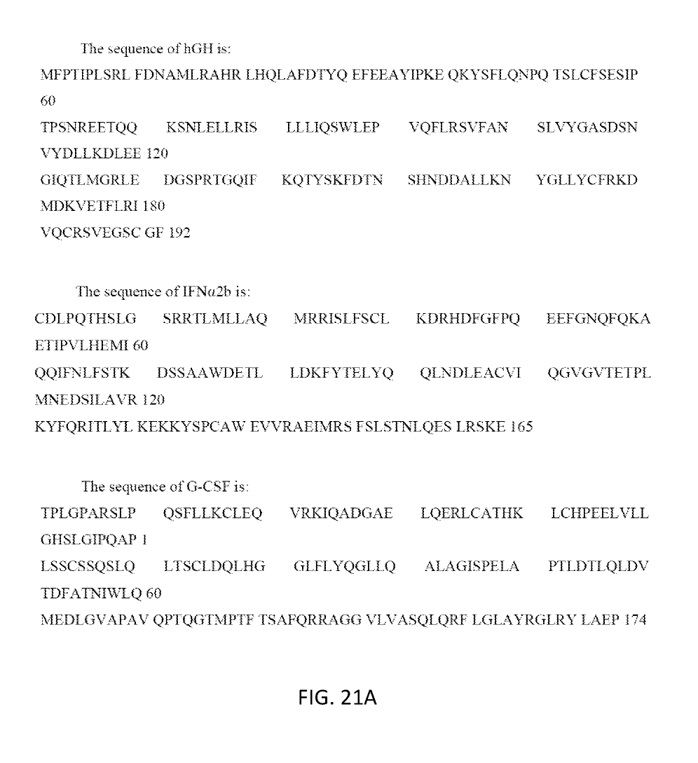

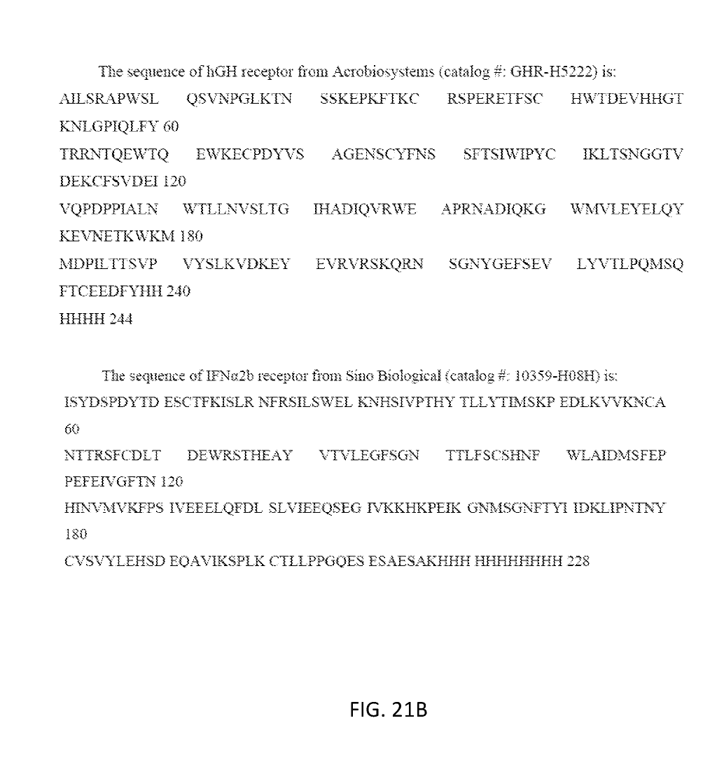

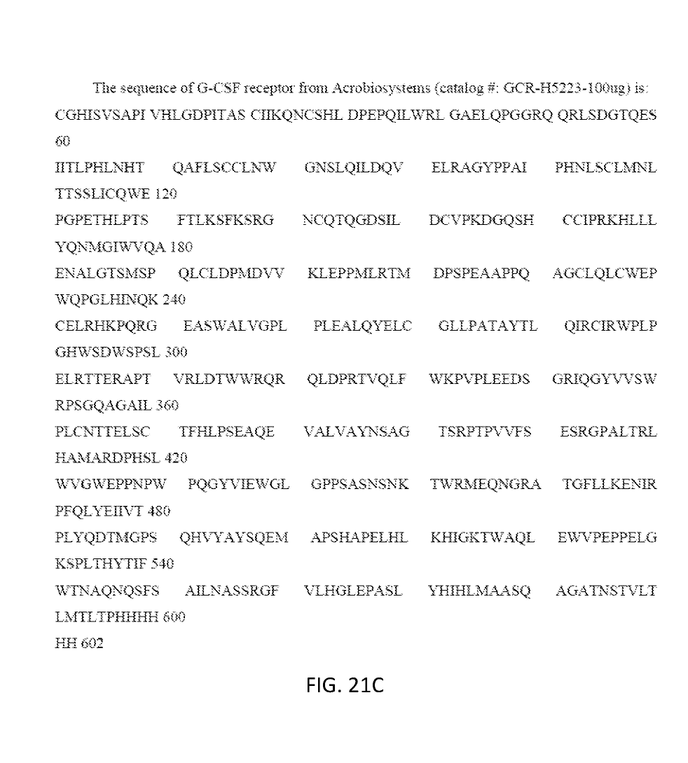

Correlation with Mass Spectrometry and Bioassay. At last, the correlation of the MCM-EC platform with industrial standard analytical technologies was studied, from the bottom level molecular characterization by mass spectrometry (MS) to the biologically relevant cellular bioassays. Same samples (Control, Medium Oxidation, and High Oxidation) of hGH and G-CSF were tested across the three technologies for comparison. MS shows that the degree of oxidation of Met residues increased from Control to Medium Oxidation and High Oxidation samples. See FIGS. 20A-20B. In the case of G-CSF, the degrees of oxidation of the Medium Oxidation sample were not directly measured. The Medium Oxidation sample was a 40%:60% mixture of High Oxidation and Control samples, which yielded intermediate degrees of oxidation.

The activity of oxidized samples was assessed by direct, competitive and degradation—determination assays in MCM-EC, all of which showed decreased activity as oxidation increased. See FIG. 20C-20D. In cell-based bioactivity assays, cellular responses and relative potency shared the same trend of decrease at higher oxidations with the MCM-EC TP-TPR assays, though the extents of decrease in bioassays were smaller. See FIG. 20E-20F. The qualitative but not quantitative correlation might be explained, considering that cellular responses are subject to a series of complicated intracellular processes after being triggered by TP-TPR binding, which may have mitigated the difference in the triggering signal. It was also observed that the Control samples had significantly lower activity than WHO Standard, which could be due to a number of factors, such as the initial oxidations in the Control samples even without artificial oxidation and inappropriate shipping and handling. Nevertheless, a good correlation was demonstrated between MCM-EC TP-TPR assays and MS and bioassay. Platforms in accordance with some embodiments of the present invention platform are capable of providing primary but similarly implicative information of TPs in a rapid and simple manner, suitable for rapid assessment of TPs produced at PoC with limited resources.

### Additional Information Concerning Examples

Reagents and Chemicals:

Unless otherwise stated, all chemicals used in the experiments were purchased from Sigma-Aldrich (St. Louis, Mo.). hGH, IFNα2b, and G-CSF samples were supplied by Sandoz Pharmaceuticals except those in the bioassay section.

In the bioassay section, hGH and G-CSF purchased from Myoderm were used for mass spectrometry, MCM-EC assays and bioassays for comparison. Stock Sandoz hGH was 10.7 mg/ml in 10 mM sodium phosphate (pH=7.0). Stock Sandoz IFNα2b was 2.58 mg/ml in 100 mM NaOAc and 250 mM NaCl (pH=4.4). Stock Sandoz G-CSF was 1.18 mg/ml 10 mM glutamic acid and 5% (w/v) sorbitol (pH=4.4). Recombinant human insulin was purchased from Life Technologies, Inc. (catalog number: 12585-014). hGH Receptor (GHR), G-CSF Receptor (GCSFR), Insulin Receptor (INR) were purchased from ACROBiosystems (catalog numbers: GHR-H5222, GCR-H5223 and INR-H5220). IFNα2b Receptor 2 (IFNAR2) was purchased from Sino Biological Inc. (catalog number: 10359—H02H).

The positive mobility modulator was a positively charged peptide (NH2-GRGR GRGR GRGR GRGR GRGR GRGR GRGR GRGR GK(5-FAM)-CONH2) (SEQ. ID. 1), which was synthesized by the Biopolymers Laboratory at Koch Institute of MIT. The N-terminus of the peptide was modified by 6-(BOChydrazino) nicotinic acid (6-BOC-HNA, catalog number: S—3003) from San Diego, Calif.) for receptor-peptide conjugation. For receptor-peptide conjugation, receptors were modified by succinimidyl 4-formylbenzoate sodium salt (Sulfo—S-4FB) crosslinker (catalog number: S-1008) from Solulink (San Diego, Calif.).

The negative mobility modulator was a 64-base oligonucleotide (5′-NH2-AG CTA GCT AGC TAG CTA GCT AGC TAG CTA GCT AGC TAG CTA GCT AGC TAG CTA GCT AGC TAG CT-3′) (SEQ. ID 2), which was synthesized by Integrated DNA Technologies (Coralville, Iowa). An Amino Modifier C12 Linker was added to the 5′ during synthesis for receptor-oligonucleotide conjugation. Receptor-oligonucleotide conjugation was realized using the protein-oligonucleotide conjugation kit (catalog number: S-9011) from Solulink (San Diego, Calif.).

ZEBA™ Spin Desalting Columns (7K MWCO, 0.5 mL) was purchased from Thermo Fisher Scientific (catalog number: 89882) for desalting during conjugation. Alexa Fluor 488 (carboxylic acid succinimidyl ester, A20000, Invitrogen) was used for fluorescence labeling of proteins. Nafion resin (20 wt % solution in lower aliphatic alcohol/H2O mix) was purchased from Sigma-Aldrich (catalog number: 527122-25ML).

Procedures of Molecular Charge Modulation (in Certain Embodiments)

Positive and negative mobility modulators were designed based on the molecular properties of the TPRs.

Positive MCM was realized by receptor-peptide conjugation. Receptor was reconstituted to 1 mg/ml in 1×PBS, followed by incubation with Sulfo-S-4FB at a molar ratio of 1:20 at room temperature for 2 hours. S-4FB modified receptor was incubated with positive mobility modulator (6-BOC-HNA modified peptide) at a molar ratio of 1:3, at 4° C. overnight.

Negative MCM was realized by receptor-oligonucleotide conjugation. The negative mobility modulator (amine-oligonucleotide) was incubated with S-4FB at a molar ratio of 1:20 at room temperature for 2 hours. Receptor was incubated with S-HyNic at a molar ratio of 1:20 at room temperature for 2.5 hours. S-HyNic modified receptor was incubated with S-4FB modified oligonucleotide at a molar ratio of 1:14, at 4° C. overnight. The HyNic-4FB conjugation chemistry yielded a UV-traceable bond, which absorbed at 354 nm and had a molar extinction coefficient of 29,000. Based on the A354 absorption and concentration of the conjugates, the degrees of conjugation (numbers of mobility modulator molecules per receptor molecule, DoCs) were determined.

Design of the Positive Mobility Modulator

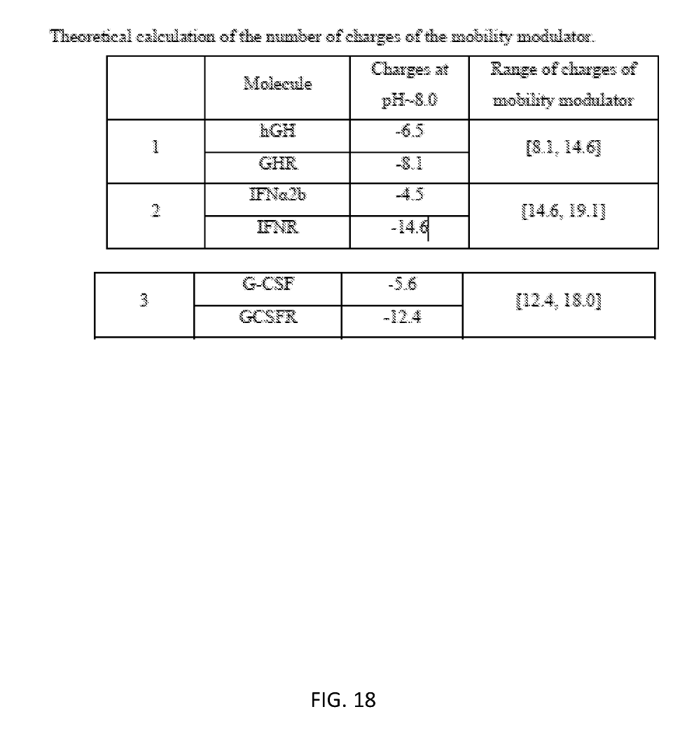

In some embodiments, for pMCM, the positively modulated receptor is designed to be positively charged and unconcentratable, while the TP-TPR complex is negatively charged and concentratable. In other words, the following relations are satisfied in these embodiments: Q(peptide)+Q(TPR)>0, Q(peptide)+Q(TPR)+Q(TP)<0. Therefore the charge of the mobility modulator, peptide, is chosen, in these embodiments, to be: -Q(TP)<Q(peptide)<-Q(TP)-Q(TPR). In some embodiments, to design a peptide with a desirable number of charges for modulating GHR, IFNR, and GCSFR, a theoretical estimation can be performed based on the charges of hGH, IFNα2b, G-CSF and their receptors. The pH near the ion depletion region in the EC device is increased to ˜8.0 due to depletion of hydrogen ions. The numbers of charges of each molecules at pH=8.0 can then be calculated using the sourceforge protein calculator v3.4 (http://protcalc.sourceforge.net). Based on the values calculated, the ranges of the charges of the mobility modulator are obtained, as depicted in the chart in FIG. 18. The number of charges that can be used for modulating all the three TP-TPR binding pairs is 14.6. Therefore, the sequence of the peptide was designed as GRGRGRGRGRGRGRGRGRGRGRGRGRGRGRGRGK(5-FAM) (SEQ ID 1), which has a net charge of ˜14.5 at pH=8.0.

Positive MCM was realized, for example, by receptor-peptide conjugation. Receptor was reconstituted to 1 mg/ml in 1×PBS, followed by incubation with Sulfo-S-4FB at a molar ratio of 1:20 at room temperature for 2 hours. S-4FB modified receptor was incubated with positive mobility modulator (6-BOC-HNA modified peptide) at a molar ratio of 1:3, at 4° C. overnight.

Design of the Negative Mobility Modulator

The EC device can sometimes have limited charge-based separation resolution: highly charged molecules (e.g. DNA) can be easily separated from minimally charged molecules (e.g. proteins), but it can be difficult to separate between molecules with slightly different mobilities (e.g. different protein species). The mobility of biomolecules can be estimated by the charge-mass ratio: Q/M, where Q is the number of charges, and M is the molecular weight in kDa. The mobility of oligonucleotides is constant, which is ˜1/0.3=3.3. The mobility of GHR, IFNR IFNα2b, and GCSF are 8.1/45=0.18, 14.6/45=0.32, 12.4/94=0.13. To sufficiently increase the mobility of receptors, an oligonucleotide with 64 bases and a molecular weight of 20 kDa was designed for conjugation. The sequence is 5′-NH2-AGCT AGCT AGCT AGCT AGCT AGCT AGCT AGCT AGCT AGCT AGCT AGCT AGCT AGCT AGCT AGCT-3′ (SEQ. ID. 2), with a C-12 amino modifier at 5′. In the EC device, it is a good estimation that molecules (the TP-TPR complex in this study) with mobility>1.0 could be easily separated from proteins (unbound TP).

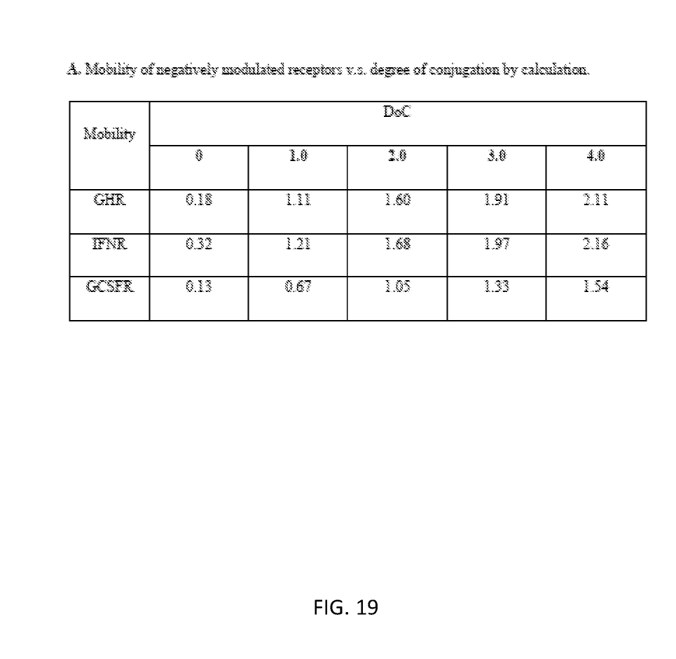

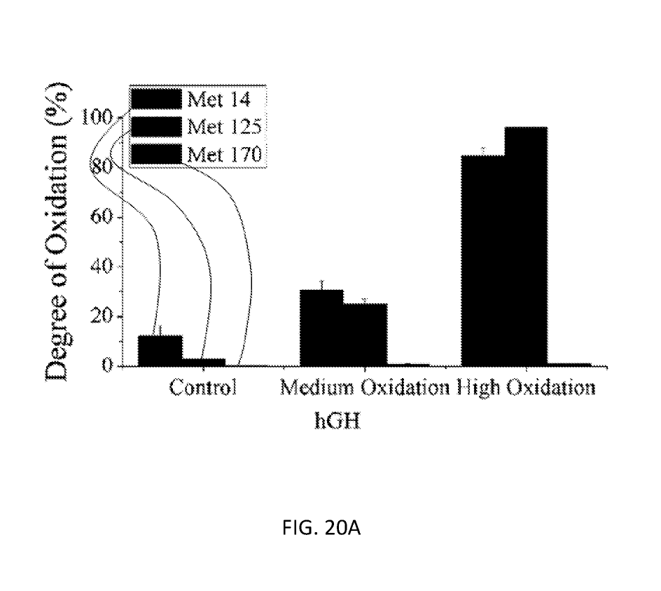

The mobility of receptors was calculated in respect to degrees of conjugation with the oligonucleotide designed (see FIG. 19). For GHR and IFNR, a minimum DoC of 1.0 was determined to separate the complex and unbound TP. For GCSFR, which is heavier, a minimum DoC of 2.0 was determined. For better separation resolution, higher DoCs can be desirable, especially if the bioactivity of the receptor is not affected due to high degrees of modification.

Determination of Degree of Conjugation (DoC)

DoC is related to the number of mobility modulator molecule per receptor molecule, which can be used for the quantification and optimization of MCM. In some embodiments, DoC was obtained by measuring the molar concentration of receptor molecule in a sample and the molar concentration of mobility modulator molecule attached to receptor molecules. The weight concentration of receptor molecule was measured by BCA protein assay and converted to molar concentration by being divided by the molecular weight of receptor. The molar concentration of attached mobility modulator molecule was represented by the molar concentration of the conjugation bond, assuming each mobility modulator molecule was attached to a receptor molecule by only one conjugation bond. In this work, the HyNic-4FB conjugation chemistry yielded a UV-traceable bond, which absorbed at 354 nm and had a molar extinction coefficient of 29,000. Through measuring the 354 nm absorption of a sample by spectrophotometry and further dividing it by the bond's molar extinction coefficient, the molar concentration of the conjugation bond and in turn that of the mobility modulator was obtained. DoC was calculated by dividing the molar concentration of mobility modulator molecule by the molar concentration of receptor molecule.

Detailed Experimental Procedures of MCM

In some embodiments, positive MCM was realized by receptor-peptide conjugation. Receptor was reconstituted to 1 mg/ml in 1×PBS, followed by incubation with Sulfo-S-4FB at a molar ratio of 1:20 at room temperature for 2 hours. After desalting the receptor with Zeba™ Spin Desalting Columns, the concentration of receptor was measured by BCA protein assay. 2 μL S-4FB modified receptor was incubated with 18 uL 0.5 mM 2-Hydrazinopyridine at 37° C. for 1 hour, which generated a compound with absorption at 350 nm. Based on the absorption measured by NanoDrop 1000 and the concentration of receptor, molar substitution ration (MSR) of S-4FB modification was determined. If MSR=4-10, there are sufficient conjugation sites for HyNic-peptide; otherwise, receptor modification should be repeated until obtaining MSR=4-10. S-4FB modified receptor was incubated with HyNic-peptide at molar ratios of 1:3, at 4° C. overnight. After desalting, the concentration of the receptor-peptide conjugate was measured by BCA protein assay. Based on the absorption at 354 nm measured by NanoDrop 1000 and the concentration of conjugate, the DoCs were determined.

In some embodiments, negative MCM was realized by receptor-oligonucleotide conjugation. Oligonucleotide was resuspended to 0.5 OD260/uL, and receptor was reconstituted to 1 mg/ml. Oligonucleotide was incubated with S-4FB at a molar ratio of 1:20 at room temperature for 2 hours. Receptor was incubated with S-HyNic at a molar ratio of 1:20 at room temperature for 2.5 hours. After desalting, the concentration of S-4FB modified oligonucleotide was measured by A260 absorption, and the concentration of S-HyNic modified receptor was measured by BCA protein assay. 2 μL S-4FB modified oligonucleotide was incubated with 18 uL 0.5 mM 2-Hydrazinopyridine at 37° C. for 1 hour, which generated a compound with absorption at 350 nm. 2 μL S-HyNic modified receptor was incubated with 18 uL 0.5 mM 2-Sulfobenzaldehyde at 37° C. for 1 hour, which generated a compound with absorption at 348 nm. Based on absorption and concentration, the MSRs of S-4FB modified oligonucleotide and S-HyNic modified receptor were determined. If the MSR of S-4FB modified oligonucleotide was ˜1.0 and the MSR of S-HyNic modified receptor was 4-10, the modifications were successful; otherwise, modifications should be repeated until obtaining correct MSRs. S-HyNic modified receptor was incubated with S-4FB modified oligonucleotide at molar ratios of 1:14, at 4° C. overnight. After desalting, the concentration of the receptor-peptide conjugate was measured by BCA protein assay. Based on the absorption at 354 nm measured by NanoDrop 1000 and the concentration of conjugate, the DoCs were determined.

Further, in some embodiments, the mixing ratios of modified receptors and peptides/oligonucleotides were adjusted to achieve optimal DoCs. S-4FB modified receptor was incubated with HyNic-peptide at molar ratios of 1:1.5, 1:3, and 1:6, at 4° C. overnight. It was observed that DoC=˜1.0 when S-4FB modified receptor:HyNic-peptide=1:3, which was the optimal condition for pMCM. S-HyNic modified receptor was incubated with S-4FB modified oligonucleotide at molar ratios of 1:3.5, 1:7.0, and 1:14, at 4° C. overnight. It was observed that DoC was higher at higher concentrations of S-4FB modified oligonucleotide. The ratio of 1:14 was adopted for nMCM.

Microchip Fabrication:

According to some embodiments, as described herein, the device was designed with five parallel channels to enable processing of five samples at the same time. Microchannels were fabricated by the standard polydimethylsiloxane (PDMS) molding technique. The silicon master for molding was fabricated by standard microfabrication processes: the desired design was patterned by photolithography onto a silicon wafer, followed by a deep reactive ion etching (DRIE) process with an etching depth of 5 The silicon master was treated overnight with trichlorosilane (T2492, UCT Specialties, Bristol, Pa.) in a vacuum desiccator to prevent PDMS adhesion to the wafer. The ion-selective Nafion nanojunction was patterned on a standard glass slide using the microflow patterning technique. A 50 μm deep and 400 μm wide microchannel was used to define the flow path of the Nafion resin. Finally, the PDMS chip was irreversibly bonded to the Nafionpatterned glass slide by plasma bonding.

Microfluidic Experiments:

In some embodiments, as described herein, before the experiment, the microchannels were passivated with 5 wt % bovine serum albumin (BSA) in 1×PBS (phosphate buffered saline) solution for 10 min to reduce nonspecific binding. The channels were flushed with 1×PBS three times before loading the samples. All assays were performed in 0.1×PBS (pH=7.4) buffer. Ag/AgCl electrodes (A-M Systems, Sequim, Wash.) were inserted into the reservoirs and connected to a DC power supply (Stanford Research Systems, Sunnyvale, Calif.). 30 V was applied in all experiments unless otherwise states. Fluorescence images were acquired using an inverted fluorescence microscope (Olympus, IX71) and a CCD camera (Sensicam qe, Cook Corp.), with an exposure time of 100 ms. All images were captured at an interval of 10 s during experiments, except in the dissociation rate measurement experiments where the interval was 30 s. A mechanical shutter was used to reduce the photobleaching effect, which was synchronized with the CCD camera by the open source software Micro-manager.

Data Analysis:

Images were analyzed using the NIH ImageJ software. A region of interest (ROI) with the width of the microchannel and a length covering the concentration plugs was selected for analysis. The intensity profile of the ROI was obtained for further analysis. The area between the profile curve and background baseline was integrated and used to represent the quantity of fluorescently labeled molecules in the concentration plug. The integral was performed using the peak analysis module of the OriginPro 9.1 software. All curve fitting shown in the figures was performed using the fitting module of the OriginPro 9.1 software.

Forced Degradation of TPs

In some embodiments, forced degradation of hGH and G-CSF for the degradation determination assay by heating, H2O2, UV exposure and longterm storage was achieved as described below:

The degradation protocols are as the following. (1) Thermal treatment of hGH and G-CSF: Before thermal treatment, hGH was diluted to 1 mg/ml using hGH stock buffer. G-CSF was used as delivered (1.13 mg/ml in G-CSF stock buffer). Five 50 uL samples of hGH were incubated in water bath for 30 min at 65° C. 75° C. 85° C., 85° C., 95° C., and 100° C., respectively. Five 50 uL samples of G-CSF were incubated in water bath for 30 min at 30° C., 40° C., 50° C., 60° C., and 70° C., respectively. All thermally treated samples were cooled down to room temperature before being used in assays. (2) Light exposure of hGH and G-CSF: Before light treatment, hGH was diluted to 1 mg/ml using hGH stock buffer. G-CSF was used as delivered (1.13 mg/ml in G-CSF stock buffer). A UV transilluminator (SPECTROLIN®, model number: TC-365R) with the range of 320 nm to 400 nm and a power of 375 Wm-2 was used to treat hGH and G-CSF samples. Four 50 uL samples of hGH and four 50 uL samples of G-CSF were exposed in the UV transilluminator for 32, 64, 128, 256 min, which had total energy of 200, 400, 800, 1600 Whm-2, respectively. (3) Oxidation of hGH and G-CSF: hGH and G-CSF were used as delivered for artificial oxidation. The oxidized samples were prepared by addition of hydrogen peroxide to give final concentrations of 0.05% (v/v) and 0.5% (v/v), and then incubation at 37° C. overnight. (4) Longterm incubation of hGH and G-CSF: Stock hGH and G-CSF were buffer exchanged to sodium borate buffer (0.1 M, pH=9). hGH samples were incubated at 37° C. for 4 weeks and 8 weeks, followed by buffer exchange back to hGH stock buffer. G-CSF samples were incubated at 37° C. for 1 day, 3 days, and 6 days.

Bioassays

In some embodiments, the High Oxidation hGH sample was prepared by addition of hydrogen peroxide to a final concentration of 0.5% (v/v), then incubated under 37° C. overnight. The High Oxidation GCSF sample was prepared by addition of hydrogen peroxide to a final concentration of 0.5% (v/v), then incubated under 37° C. for 2 hours. The Medium Oxidation samples of hGH and GCSF were 40%:60% mixtures of the High Oxidation and Control samples. Details of sample preparation are provided below:

hGH (HUMATROPE®, Lilly, USA) were initially reconstituted in sample buffer (10 mM sodium phosphate, pH=7) to a concentration of 2 mg/mL. The High Oxidation hGH sample was prepared by addition of hydrogen peroxide to a final concentration of 0.5% (v/v), then incubated under 37° C. overnight. After the incubation, the High Oxidation hGH was dialyzed back into sodium phosphate sample buffer, to avoid further oxidation. G-CSF, with an initial concentration at 0.6 mg/mL (NEUPOGEN®, Amgen, USA) were dialyzed into sample buffer (20 mM glutamic acid with 5% sorbitol, w/v, pH=4.4). The High Oxidation G-CSF sample was prepared by addition of hydrogen peroxide to a final concentration of 0.5% (v/v), then incubated under 37° C. for 2 hours. The High Oxidation G-CSF was dialyzed back into glutamic acid sample buffer. Dialyzation was performed with 200 μL buffer at 13,000 rpm for 20 minutes in a 500 μL 10 kDa Amicon (EMD Millipore Corporation, Merck, Germany) centrifugal filter, which was repeated three times. The Medium Oxidation samples of hGH and G-CSF were 40%:60% mixtures of the High Oxidation and Control samples. Control, Medium Oxidation and High Oxidation samples were all lyophilized overnight. Upon testing, all samples were reconstituted to a final concentration of 2 mg/mL of hGH and 0.6 mg/mL of G-CSF, and stored at −80° C. for further use.

The measurement of degrees of oxidation by mass spectrometry is described below: 50 μg hGH sample was dissolved in 6M guanidinium chloride and reduced by 10 mM dithiothreitol under 70° C. for 30 minute, which was followed by alkylation with 55 mM iodoacetamide under room temperature in dark condition. Proteins were dialyzed to Tris-HCl buffer (pH=6.8) with a 10 kD membrane Amicon centrifugal filter at 13,000 rmp for 15 minutes, which was repeated three times. The following in-solution digestion process with Trypsin was kept overnight at room temperature to avoid artificial oxidation. The digestion was terminated by addition of 20 μL 5% formic acid. 50 μg G-CSF sample was adjusted to a pH of 3 by HCl, followed by digestion with pepsin at 37° C. for 30 minutes. The digestion process was terminated by adjusting the pH to 8 by 0.1M NH4CO3. Proteins were then ready for LC-MS analysis, the remaining materials were aliquot to 20 μL and stored in −80° C. for further analysis. LC-MS analysis used an Ultimate 3000 nano LC pump (Dionex, Mountain View, Calif.) and selfpacked C18 column (Magic C18, 200A pore and 5 μm particle size, 75 μm internal diameter by 100 mm) connected to a coated emitter with an internal diameter of 10 μm (New Objective, Woburn, Mass.). LTQ-Orbitrap XL mass spectrometer (Thermo Fisher Scientific, San Jose, Calif.) was connected through a nanospray ion source (New Objective, Woburn, Mass.). 0.1% formic acid in HPLC grade water was used as Mobile Phase A and 0.1% formic acid in acetonitrile was used as Mobile Phase B. During sample injection, the flow rate was set to 250 nL/min with 2% B for 25 min. The flow rate of the gradient was set to 200 nL/min, with mobile phase B, 0-60 min 40%, 60-70 min 90%, 70-75 min 90% and 75-78 min 2%. The mass spectrometer was operated in a data dependent mode to switch between MS and CID-MK. Briefly, after a full-scan MS spectrum from m/z 400-2000 in the ion-trap, 8 CID-MS2 activation steps were performed on the 8 most intense precursor ions from the full scan. All control and variants samples were run in triplicate. For peptide identification, raw data were searched against human growth hormone and granulocyte-colony stimulating factor sequence using SEQUEST incorporated in Proteomic Discover 1.4 (Thermo Fisher Scientific). Peptide precursor ion mass tolerance was set to 1.0 Da, and the fragment ion mass tolerance 1.0 Da. Oxidation of Methionine residues were set as a potential dynamic modification. The identified peptides were then filtered using Xcorr score (1+precursor ion >1.9, 2+>2.2, and 3+ and above >3.4). Mass accuracy was set to <50 ppm. Final confirmation of the peptide identification was determined by manual inspection, extracting the base peak from the chromatogram and matching the MS2 fragmentation data with theoretical prediction. The oxidation percentage was calculated by peptide peak area. Bioassays of hGH and G-CSF were conducted by Bioassay GmbH (Heidelberg, Germany), which is an independent contract laboratory certified by good laboratory practice (GLP) regulations and good manufacturing practice (GMP) regulations. For hGH, the potency assay was performed with NB2-11 cell line by measuring the proliferation of cells. For G-CSF, the potency assay was performed with NFS 60 cell line by measuring the tetrazolium conversion of cells. Bioassays were conducted in three separate plates, each of which included three separate experiments, yielding a total of nine sets of data for each sample. The relative potency of each sample was calculated based on the mean and standard deviation of the full nine sets of data.

Additional Information:

Human growth hormone (hGH) experiments. For the direct assay, 10 nM positively modulated hGH receptor (GHR(+)) and 0-243 nM hGH were incubated 30 minutes before testing. For the competitive assay, 200 nM negatively modulated hGH receptor (GHR(−)), 200 nM labeled hGH and 0-3200 nM target hGH were incubated 30 min.

Kinetics experiments: For off-rate measurement, 100 nM labeled drug (hGH/IFN a2b/G-CSF/insulin) and 1 uM negatively modulated receptor (GHR/IFNR/GCSFR/INR) were incubated for 30 minutes.

The teachings of all patents, published applications and references cited herein are incorporated by reference in their entirety.

While this invention has been particularly shown and described with references to example embodiments thereof, it will be understood by those skilled in the art that various changes in form and details may be made without departing from the scope of the invention encompassed by the appended claims.

What is claimed is:

1. A method for assaying an analyte using a mobility based assay comprising:
a) providing an analyte and a receptor capable of binding to the analyte to form a complex, the analyte, the receptor, and the receptor-analyte complex having respective mobilities;
b) modifying the receptor to modulate its mobility;
c) combining the analyte and the modified receptor to form a modified receptor-analyte complex; and
d) assaying the combination of the analyte and modified receptor using a mobility based assay to separate species along at least one separation dimension and to detect at least one of the analyte, the modified receptor, and the modified receptor-analyte complex at a respective location along the separation dimension.

2. The method of claim 1, wherein the assaying includes determining at least one of an equilibrium and a kinetic binding parameter for the analyte.

3. The method of claim 1, wherein the mobility of the modified receptor has a directionality opposite to that of a mobility of the modified receptor-analyte complex.

4. The method of claim 3, wherein the mobility of the receptor has the same directionality as that of the mobility of the receptor-analyte complex.

5. The method of claim 4, wherein the mobility based assay is further used to electrokinetically concentrate the modified receptor-analyte complex.

6. The method of claim 5, wherein the assay does not provide for an electrokinetic concentration of the receptor in an uncomplexed state.

7. The method of claim 1, wherein the receptor is provided with a detectable label.

8. The method of claim 1, wherein the analyte is a biologic.

9. The method of claim 1, wherein the analyte is a drug.

10. The method of claim 1, wherein the mobility of the modified receptor is greater than, and in the same direction as, the mobility of the receptor.

11. The method of claim 10, wherein the mobility of the modified receptor-analyte complex is greater than the mobility of the drug.

12. The method of claim 11, wherein the location of the modified receptor-analyte complex along the separation dimension is resolvable from the location of the analyte.

13. The method of claim 12, wherein the assaying includes determining at least one of an equilibrium and a kinetic binding parameter for the analyte.

14. The method of claim 12, further comprising providing a labeled analyte, wherein the assaying includes competition between the labeled analyte and the analyte for receptor binding.

15. The method of claim 10, wherein the analyte is a biologic.

16. The method of claim 10, wherein the analyte is a drug.

17. A method of determining the activity of a drug comprising:
a) using charge polarity transition or mobility enhancement in a mobility-based assay to determine at least one of a kinetic and an equilibrium binding parameter for a drug-receptor interaction;
b) obtaining a reference value for the at least one of a kinetic and an equilibrium binding parameter; and
c) comparing the determined parameter with the reference value to determine drug activity.

18. A microfluidic drug-receptor binding assay for analyte activity assessment comprising:
a) a receptor conjugated to a charged modulator to form a modulated receptor, the modulated receptor having a net charge, the net charge having a polarity; and
b) an analyte;
c) wherein, a binding of the analyte to the modulated receptor forms a complex having a net charge, the net charge on the complex having a polarity that is opposite to the polarity of the net charge on the modulated receptor.


SEQUENCE LISTING (8 sequences)

SEQ ID NO: 1 - protein, 34 aa, Artificial Sequence
  Positive Charge Modulator
GRGRGRGRGRGRGRGRGRGRGRGRGRGRGRGRGK

SEQ ID NO: 2 - protein, 64 aa, Artificial Sequence
  Negative Charge Modulator
AGCTAGCTAGCTAGCTAGCTAGCTAGCTAGCTAGCTAGCTAGCTAGCTAGCTAGCTAGCTAGCT

SEQ ID NO: 3 - protein, 192 aa, Homo sapien
MFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 4 - protein, 165 aa, Homo sapien
CDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMNEDSILAVRKYFQRITLYLKEKKYSPCAWEVVRAEIMRSFSLSTNLQESLRSKE

SEQ ID NO: 5 - protein, 174 aa, Homo sapien
TPLGPARSLPQSFLLKCLEQVRKIQADGAELQERLCATHKLCHPEELVLLGHSLGIPQAPLSSCSSQSLQLTSCLDQLHGGLFLYQGLLQALAGISPELAPTLDTLQLDVTDFATNIWLQMEDLGVAPAVQPTQGTMPTFTSAFQRRAGGVLVASQLQRFLGLAYRGLRYLAEP

SEQ ID NO: 6 - protein, 244 aa, Homo sapien
AILSRAPWSLQSVNPGLKTNSSKEPKFTKCRSPERETFSCHWTDEVHHGTKNLGPIQLFYTRRNTQEWTQEWKECPDYVSAGENSCYFNSSFTSIWIPYCIKLTSNGGTVDEKCFSVDEIVQPDPPIALNWTLLNVSLTGIHADIQVRWEAPRNADIQKGWMVLEYELQYKEVNETKWKMMDPILTTSVPVYSLKVDKEYEVRVRSKQRNSGNYGEFSEVLYVTLPQMSQFTCEEDFYHHHHHH

SEQ ID NO: 7 - protein, 228 aa, Homo sapien
ISYDSPDYTDESCTFKISLRNFRSILSWELKNHSIVPTHYTLLYTIMSKPEDLKVVKNCANTTRSFCDLTDEWRSTHEAYVTVLEGFSGNTTLFSCSHNFWLAIDMSFEPPEFEIVGFTNHINVMVKFPSIVEEELQFDLSLVIEEQSEGIVKKHKPEIKGNMSGNFTYIIDKLIPNTNYCVSVYLEHSDEQAVIKSPLKCTLLPPGQESESAESAKHHHHHHHHHHH

SEQ ID NO: 8 - protein, 602 aa, Homo sapien
CGHISVSAPIVHLGDPITASCIIKQNCSHLDPEPQILWRLGAELQPGGRQQRLSDGTQESIITLPHLNHTQAFLSCCLNWGNSLQILDQVELRAGYPPAIPHNLSCLMNLTTSSLICQWEPGPETHLPTSFTLKSFKSRGNCQTQGDSILDCVPKDGQSHCCIPRKHLLLYQNMGIWVQAENALGTSMSPQLCLDPMDVVKLEPPMLRTMDPSPEAAPPQAGCLQLCWEPWQPGLHINQKCELRHKPQRGEASWALVGPLPLEALQYELCGLLPATAYTLQIRCIRWPLPGHWSDWSPSLELRTTERAPTVRLDTWWRQRQLDPRTVQLFWKPVPLEEDSGRIQGYVVSWRPSGQAGAILPLCNTTELSCTFHLPSEAQEVALVAYNSAGTSRPTPVVFSESRGPALTRLHAMARDPHSLWVGWEPPNPWPQGYVIEWGLGPPSASNSNKTWRMEQNGRATGFLLKENIRPFQLYEIIVTPLYQDTMGPSQHVYAYSQEMAPSHAPELHLKHIGKTWAQLEWVPEPPELGKSPLTHYTIFWTNAQNQSFSAILNASSRGFVLHGLEPASLYHIHLMAASQAGATNSTVLTLMTLTPHHHHHH